SEQUENCE LISTING (33 sequences)

SEQ ID NO: 1 - protein, 10 aa, synthetic construct
GRTFSNYAMG

SEQ ID NO: 2 - protein, 17 aa, synthetic construct
RISGGGGYTAYADSVKG

SEQ ID NO: 3 - protein, 17 aa, synthetic construct
GSIDSRQPYDSTRRYDY

SEQ ID NO: 4 - protein, 9 aa, synthetic construct
RASTYIAMA

SEQ ID NO: 5 - protein, 17 aa, synthetic construct
AITWSGGHTTYADSMKG

SEQ ID NO: 6 - protein, 15 aa, synthetic construct
NQRNTVGPSEGAYPY

SEQ ID NO: 7 - protein, 10 aa, synthetic construct
GRTLSNHAMH

SEQ ID NO: 8 - protein, 17 aa, synthetic construct
AITWSDGETYYEDSVKG

SEQ ID NO: 9 - protein, 14 aa, synthetic construct
KMGGPTSIPGLVEY

SEQ ID NO: 10 - protein, 10 aa, synthetic construct
GRTLSNHAMH

SEQ ID NO: 11 - protein, 17 aa, synthetic construct
AIPRSGSNIGYSAFVKD

SEQ ID NO: 12 - protein, 20 aa, synthetic construct
KSAAGYYSGVVFTADYDYTY

SEQ ID NO: 13 - protein, 10 aa, synthetic construct
GLTFSVYRMG

SEQ ID NO: 14 - protein, 17 aa, synthetic construct
AISRIADSTYYADSVKG

SEQ ID NO: 15 - protein, 17 aa, synthetic construct
GSRVFDSRWYDVNEYYY

SEQ ID NO: 16 - protein, 160 aa, synthetic construct
EVQLVESGGGLVQAGGSLSVSCAPSGRTFSNYAMGWFRQAPGKEREFVARISGGGGYTAYADSVKGRFTIARDNAKNTVYLQMNSLKPEDTAVYYCAAGSIDSRQPYDSTRRYDYWGQGTLVTVSSAAADYKDHDGDYKDHDIDYKDDDDKGAAHHHHHH

SEQ ID NO: 17 - protein, 158 aa, synthetic construct
EVQLVESGGGLKQAGGSLRLSCTASARASTYIAMAWFRRTPGKAREFVAAITWSGGHTTYADSMKGRFTISRDNAKNTVYLHLNALQPEDAGVYYCAANQRNTVGPSEGAYPYWGQGTLVTVSSAAADYKDHDGDYKDHDIDYKDDDDKGAAHHHHHH

SEQ ID NO: 18 - protein, 157 aa, synthetic construct
EVQLVESGGGLVQPGGSLRLSCAASGRTLSNHAMHWFRQAPGKEREFVSAITWSDGETYYEDSVKGRFTISRDNAKDTAYLEMQSLKPEDTAVYYCAAKMGGPTSIPGLVEYWGQGTLVTVSSAAADYKDHDGDYKDHDIDYKDDDDKGAAHHHHHH

SEQ ID NO: 19 - protein, 163 aa, synthetic construct
EVQLVESGGGLVQAGDSLRLSCVASGRTFSSYHMGWFRQAPGKEREFVAAIPRSGSNIGYSAFVKDRGTISRDNAKNTVYLQINNLAPDDTAVYYCAAKSAAGYYSGVVFTADYDYTYWGQGTLVTVSSAAADYKDHDGDYKDHDIDYKDDDDKGAAHHHHHH

SEQ ID NO: 20 - protein, 489 aa, synthetic construct
MGWSCIILFLVATATGVHSEVQLVESGGGLVQAGGSLSVSCAPSGRTFSNYAMGWFRQAPGKEREFVARISGGGGYTAYADSVKGRFTIARDNAKNTVYLQMNSLKPEDTAVYYCAAGSIDSRQPYDSTRRYDYWGQGTLVTVSSAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGGCLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPEGHHHHHHG

SEQ ID NO: 21 - protein, 487 aa, synthetic construct
MGWSCIILFLVATATGVHSEVQLVESGGGLKQAGGSLRLSCTASARASTYIAMAWFRRTPGKAREFVAAITWSGGHTTYADSMKGRFTISRDNAKNTVYLHLNALQPEDAGVYYCAANQRNTVGPSEGAYPYWGQGTLVTVSSAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGGCLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPEGHHHHHHG

SEQ ID NO: 22 - protein, 486 aa, synthetic construct
MGWSCIILFLVATATGVHSEVQLVESGGGLVQPGGSLRLSCAASGRTLSNHAMHWFRQAPGKEREFVSAITWSDGETYYEDSVKGRFTISRDNAKDTAYLEMQSLKPEDTAVYYCAAKMGGPTSIPGLVEYWGQGTLVTVSSAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGGCLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPEGHHHHHHG

SEQ ID NO: 23 - protein, 492 aa, synthetic construct
MGWSCIILFLVATATGVHSEVQLVESGGGLVQAGDSLRLSCVASGRTFSSYHMGWFRQAPGKEREFVAAIPRSGSNIGYSAFVKDRGTISRDNAKNTVYLQINNLAPDDTAVYYCAAKSAAGYYSGVVFTADYDYTYWGQGTLVTVSSAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGGCLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPEGHHHHHHG

SEQ ID NO: 24 - protein, 19 aa, synthetic construct
MGWSCIILFLVATATGVHS

SEQ ID NO: 25 - protein, 231 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGG

SEQ ID NO: 26 - protein, 102 aa, synthetic construct
CLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPE

SEQ ID NO: 27 - protein, 8 aa, synthetic construct
GHHHHHHG

SEQ ID NO: 28 - protein, 233 aa, synthetic construct
MGWSCIILFLVATATGVHSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTNQGLSSPVTKSFNRGEC

SEQ ID NO: 29 - protein, 109 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRT

SEQ ID NO: 30 - protein, 105 aa, synthetic construct
VAAPSVFIFPPSDEQLKSGTASVVCLLNNFYPREAKVQWKVDNALQSGNSQESVTEQDSKDSTYSLSSTLTLSKADYEKHKVYACEVTNQGLSSPVTKSFNRGEC

SEQ ID NO: 31 - protein, 581 aa, synthetic construct
MGWSCIILFLVATATGVHSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGGCLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPEGHHHHHHG

SEQ ID NO: 32 - protein, 118 aa, synthetic construct [sequence omitted]

SEQ ID NO: 33 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKASGG

(19) United States
(12) Patent Application Publication
(10) Pub. No.: US 2025/0034258 A1
(43) Pub. Date: Jan. 30, 2025

(54) BLOCKING AND NON-BLOCKING SINGLE DOMAIN ANTIBODIES AND USES THEREOF

(71) Applicant: Merck Sharp & Dohme LLC, Rahway, NJ (US)

(72) Inventors: Edward P. Bowman, Redwood City, CA (US); Olugbeminiyi O. Fadeyi, Harvard, MA (US); Rob C. Oslund, Brookline, MA (US)

(73) Assignee: Merck Sharp & Dohme LLC, Rahway, NJ (US)

(21) Appl. No.: 18/714,178

(22) PCT Filed: Dec. 1, 2022
(86) PCT No.: PCT/US2022/051488

§ 371 Date: May 29, 2024

Related U.S. Application Data
(60) Provisional application No. 63/285,520, filed on Dec. 3, 2021.

(51) Int. Cl. C07K 16/28; G01N 33/50; G01N 33/58

(52) U.S. Cl. CPC C07K 16/2827; C07K 16/2818; G01N 33/5032; G01N 33/58; C07K 2317/565; C07K 2317/569; C07K 2317/76; C07K 2319/02; C07K 2319/21

(57) ABSTRACT

The invention relates, in part, to single domain antibody constructs. In particular, the invention relates, in part, to single domain antibody constructs that include a non-blocking α-PDL1 VHH that would provide minimal disruption of the PD-1/−PD-L1 binding interaction. The invention further relates to a method for detecting cell-cell interactions using the VHH sequences provided herein.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jul. 21, 2022, is named 25145-WO-PCT_SL.XML and is 33 KB in size.

### FIELD OF THE INVENTION

The present invention relates, in part, to single domain antibody constructs. In particular, the present invention relates, in part, to single domain antibody constructs that include non-blocking and blocking α-PD-L1 VHH and α-PD-1 VHH that would provide minimal disruption of the PD-1/−PD-L1 binding interaction. Other aspects, embodiments, features, uses and advantages of the invention will be clear to the skilled person based on the disclosure herein.

### BACKGROUND OF THE INVENTION

Direct cell-cell interactions play an essential role in the development and biological function of all multicellular organisms. These interactions occur through protein-, glycan-, and/or lipid-mediated physical contact at the plasma membrane to form cellular junctions, adhesions, or synapses in either stable or transient fashion. These critical interactions drive the organization of cells into tissues and other complex cellular environments with important consequences on human health. A key example is the tumor microenvironment where interactions between cancer, endothelial, stromal, and immune cells dictate tumor progression. Within this context, T cells form immunological synapses with antigen presenting cells (APCs) through binding events between the T cell Receptor (TCR) and the peptide MHC complex (pMHC), adhesion molecules, and costimulatory/checkpoint receptor-ligand pairs on T cell and APC cell surfaces. The precise spatial and temporal organization of these surface proteins at the T cell-APC interface governs critical immunological signaling events to determine tumor immune response outcomes. The growing appreciation for the role of T cells in driving tumor immune responses has spurred significant efforts in developing checkpoint inhibitor and adoptive T cell transfer therapies. This has led to an increased need to develop technologies that gain insight into cellular contacts formed between immune cells and their cellular targets.

However, probing interactions within a cellular synapse is a difficult task. The spatially restricted distance of the synapse and the plethora of cell types and proteins involved places a significant burden on identifying suitable technologies that can adequately and broadly profile within this environment. Although recent enzyme-based proximity labeling approaches have been used to interrogate intercellular interactions and their environments, these strategies rely on genetic manipulation or presence of surface glycans to introduce cell tagging enzymes. Thus, a simple, modular, and non-cell perturbing approach that can readily localize a labeling cargo to a targeting protein of interest within a cellular junction is needed. Specifically, a targeting modality driven photocatalytic-based protein tagging technology that facilitates generation of longer-lived reactive species for capturing cell-cell interactions is needed.

### SUMMARY OF THE INVENTION

The present invention provides a binding protein comprising a heavy chain antibody variable domain (VHH) that specifically binds to PD-1 or PD-L1, wherein the VHH comprises three complementarity determining regions (CDRs) selected from the group consisting of:
a) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3);
b) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6);
c) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9);
d) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and
e) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15).

In some embodiments, the present invention provides a heavy chain antibody variable domain (VHH) that specifically binds to PD-1 or PD-L1, comprising
(a) a heavy chain variable region having at least 90% amino acid sequence identity to the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16);
(b) a heavy chain variable region having at least 90% amino acid sequence identity to the amino acid sequence: EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17);
(c) a heavy chain variable region having at least 90% amino acid sequence identity the amino acid sequence: EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or
(d) a heavy chain variable region having at least 90% amino acid sequence identity to the amino acid sequence: EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

In some embodiments, a binding protein is provided comprising a heavy chain antibody variable domain (VHH) that specifically binds to PD-1 or PD-L1, comprising:
a) the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16);
b) the amino acid sequence EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17);
c) the amino acid sequence EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or
d) the amino acid sequence EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

In another embodiment, a VHH-Fc fusion protein is provided comprising the amino acid sequence:

a)
(SEQ ID NO: 20)
MGWSCIILFLVATATGVHSEVQLVESGGGLVQAGGSLSVSCAPSG
RTFSNYAMGWFRQAPGKEREFVARISGGGGYTAYADSVKGRFTIA
RDNAKNTVYLQMNSLKPEDTAVYYCAAGSIDSRQPYDSTRRYDYW
GQGTLVTVSSAAADKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKASGGCLSYDTEVLTVEYGFVPIGEIVDKGI
ECSVFSIDSNGIVYTQPIAQWHHRGKQEVFEYCLEDGSIIKATKD
HKFMTQDGKMLPIDEIFEQELDLLQVKGLPEGHHHHHHG;
b)
(SEQ ID NO: 21)
MGWSCIILFLVATATGVHSEVQLVESGGGLKQAGGSLRLSCTASA
RASTYIAMAWFRRTPGKAREFVAAITWSGGHTTYADSMKGRFTIS
RDNAKNTVYLHLNALQPEDAGVYYCAANQRNTVGPSEGAYPYWGQ
GTLVTVSSAAADKTHTCPPCPAPELLGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPAPIEKTISKAKGQPR
EPQVYTLPPSREEMTKNQVSLTCLVKGFYPSDIAVEWESNGQPEN
NYKTTPPVLDSDGSFFLYSKLTVDKSRWQQGNVFSCSVMHEALHN
HYTQKSLSLSPGKASGGCLSYDTEVLTVEYGFVPIGEIVDKGIEC
SVFSIDSNGIVYTQPIAQWHHRGKQEVFEYCLEDGSIIKATKDHK
FMTQDGKMLPIDEIFEQELDLLQVKGLPEGHHHHHHG;
c)
(SEQ ID NO: 22)
MGWSCIILFLVATATGVHSEVQLVESGGGLVQPGGSLRLSCAASG
RTLSNHAMHWFRQAPGKEREFVSAITWSDGETYYEDSVKGRFTIS
RDNAKDTAYLEMQSLKPEDTAVYYCAAKMGGPTSIPGLVEYWGQG
TLVTVSSAAADKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSREEMTKNQVSLTCLVKGFYPSDIAVEWESNGQPENN
YKTTPPVLDSDGSFFLYSKLTVDKSRWQQGNVFSCSVMHEALHNH
YTQKSLSLSPGKASGGCLSYDTEVLTVEYGFVPIGEIVDKGIECS
VFSIDSNGIVYTQPIAQWHHRGKQEVFEYCLEDGSIIKATKDHKF
MTQDGKMLPIDEIFEQELDLLQVKGLPEGHHHHHHG;
or
d)
(SEQ ID NO: 23)
MGWSCIILFLVATATGVHSEVQLVESGGGLVQAGDSLRLSCVASG
RTFSSYHMGWFRQAPGKEREFVAAIPRSGSNIGYSAFVKDRGTIS
RDNAKNTVYLQINNLAPDDTAVYYCAAKSAAGYYSGVVFTADYDY
TYWGQGTLVTVSSAAADKTHTCPPCPAPELLGGPSVFLFPPKPKD
TLMISRTPEVTCVVVDVSHEDPEVKFNWYVDGVEVHNAKTKPREE
QYNSTYRVVSVLTVLHQDWLNGKEYKCKVSNKALPAPIEKTISKA
KGQPREPQVYTLPPSREEMTKNQVSLTCLVKGFYPSDIAVEWESN
GQPENNYKTTPPVLDSDGSFFLYSKLTVDKSRWQQGNVFSCSVMH
EALHNHYTQKSLSLSPGKASGGCLSYDTEVLTVEYGFVPIGEIVD
KGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, a method of preparing a VHH conjugate is provided comprising the steps of: a) providing a VHH; b) optionally, providing a fusion protein; c) providing an intein; and d) fusing the intein to the C-terminus of the VHH to form a VHH-intein conjugate or, optionally, fusing the intein to the C-terminus of the fusion protein to form a VHH-fusion protein-intein conjugate, wherein the VHH comprises a sequence as set forth in any of the embodiments herein.

In some embodiments, a method for detecting cell-cell interactions is provided, the method comprising: a) contacting a first unlabeled cell with a VHH-photocatalyst conjugate to form a first labeled cell; b) providing a second unlabeled cell; c) combining the first labeled cell and the second unlabeled cell to form a cell-cell conjugate system; d) applying visible light in the range of approximately about 400-800 nm to the cell-cell conjugate system; e) administering a labeling probe to the cell-cell conjugate system; wherein the labeling probe covalently attaches to the first labeled cell and the second unlabeled cell; f) observing the labeling probe in detection of the cell-cell interaction, wherein the VHH-photocatalyst conjugate comprises a VHH having an amino acid sequence as set forth herein.

### BRIEF DESCRIPTION OF THE DRAWINGS

FIGS. 1A and 1B depict a graphical representation of the binding of α-PD-L1 VHH, α-PD-L1 VHH 2,and α-PD-L1 VHH 3 to CHO-K1.hPD-L1 expression cells.

FIGS. 2A and 2B depict a graphical representation of PD-1-Fc mediated blockade of anti-PD-L1 antibody, α-PD-L1 VHH, α-PD-L1 VHH 2, and α-PD-L1 VHH3 binding to CHO-K1.hPD-L1 expression cells.

FIG. 3 depicts a graphical representation of α-PD-L1 VHH, α-PD-L1 VHH 2, or α-PD-L1 VHH 3 mediated blockade of human CD80-Fc binding to biotinylated human PD-L1-Fc.

FIG. 4 is a graphical representation of the binding of α-PD-1 VHH to CHO-K1.hPD-1 expression cells.

FIG. 5 is a graphical representation of −PD-1 VHH mediated blockade of PD-L1-Fc binding to CHO-K1.hPD-L1 expression cells.

FIGS. 6A-6E represent Coomassie stained SDS-PAGE analysis of purified Isotype-VHH-Fc-2N3 (6A), PD-L1-VHH-FC-2N3 (6B), PD-L1-VHH 2-FC-2N3 (6C), PD-L1-VHH 3-FC-2N3 (6D), and PD-1-VHH-FC-2N3 (6E) under reducing (+β-ME) and non-reducing (−β-ME) conditions

FIGS. 7A and 7B depict RP-HPLC and ESI-MS analysis under reducing conditions of bis-azido conjugates.

FIGS. 8A and 8B depict a graphical representation of RP-HPLC and ESI-MS analysis under reducing conditions of bis-azido conjugates.

FIGS. 9A and 9B depict graphical representation of ESI-MS analysis under non-reducing conditions.

FIGS. 10A, 10B, and 10C depict flow cytometry analysis shows cell surface binding of PD-L1 VHH-Fc (α-PD-L1 VHH-Fc labeled with 488 dye (α-PD-L1 VHH-Fc-488)) only on PD-L1 expressing cells.

FIG. 11 is a graphical representation showing that α-PD-L1 VHH-Fc does not block the PD-1/PD-L1 interaction.

FIGS. 12A and 12B is a graphical representation showing the detection of PD-L1 in the immune synapse using α-PD-L1 VHH-Fc.

FIGS. 13A and 13B represent confocal microscopy imaging of Jurkat PD-1/JY PD-L1 and Jurkat PD-1/CHO PD-L1 two cell systems with α-PD-L1 VHH-Fc-RFT-targeted biotinylation on JY PD-L1.

### DETAILED DESCRIPTION OF THE INVENTION

### Abbreviations

Throughout the detailed description and examples of the invention the following abbreviations will be used:
ADCC Antibody-dependent cellular cytotoxicity
CDC Complement-dependent cytotoxicity
CDR Complementarity determining region in the immunoglobulin variable regions, defined using the Kabat numbering system
CHO Chinese hamster ovary
DTT Dithiothreitol
EC50 concentration resulting in 50% efficacy or binding
ELISA Enzyme-linked immunosorbent assay
FR Antibody framework region: the immunoglobulin variable regions excluding the CDR regions.
HRP Horseradish peroxidase
IC50 concentration resulting in 50% inhibition
IgG Immunoglobulin G
Kabat An immunoglobulin alignment and numbering system pioneered by Elvin A. Kabat ((1991) Sequences of Proteins of Immunological Interest, 5th Ed. Public Health Service, National Institutes of Health, Bethesda, Md.)
mAb or Mab or MAb Monoclonal antibody
MES 2-(N-morpholino)ethanesulfonic acid
PCR Polymerase chain reaction
PK Pharmacokinetics
RCF Relative centrifugal field
RFT Riboflavin tetraacetate
SED Staphylococcus Enterotoxin D
V region The segment of IgG chains which is variable in sequence between different antibodies. It extends to Kabat residue 109 in the light chain and 113 in the heavy chain.
VH Immunoglobulin heavy chain variable region
VHH Heavy chain single domain antibodies
VL Immunoglobulin kappa light chain variable region

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activation” as it applies to cells or to receptors refers to the activation or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including KinExA and Biacore. Specific illustrative and exemplary embodiments for measuring binding affinity are described in the following.

As used herein, the term “antibody,” “immunoglobulin,” or “Ig,” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies.

As used herein, unless otherwise indicated, “antigen binding fragment,” “antigen binding domain,” or “antigen binding region” refers to the portion of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments.

A “Fab fragment” is comprised of one light chain and the CHI and variable regions of one heavy chain. The heavy chain of a Fab molecule cannot form a disulfide bond with another heavy chain molecule. An “Fab fragment” can be the product of papain cleavage of an antibody.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

An “Fv fragment” or “Fv region” comprises the variable regions from both the heavy and light chains, but lacks the constant regions.

“Isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

The term “monoclonal antibody,” as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, a “chimeric antibody” is an antibody having the variable domain from a first antibody and the constant domain from a second antibody, where the first and second antibodies are from different species. (U.S. Pat. No. 4,816,567; and Morrison et al., (1984) Proc. Natl. Acad. Sci. USA 81: 6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g. rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” or “human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J. Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table I.

TABLE I
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “epitope”, as used herein, is defined in the context of a molecular interaction between an “antigen binding molecule”, such as an antibody (Ab), and its corresponding “antigen” (Ag). Generally, “epitope” refers to the area or region on an Ag to which an Ab specifically binds, i.e., the area or region in physical contact with the Ab. Physical contact may be defined through distance criteria (e.g. a distance cut-off of 4 Å) for atoms in the Ab and Ag molecules.

The epitope for a given antibody (Ab)/antigen (Ag) pair can be defined and characterized at different levels of detail using a variety of experimental and computational epitope mapping methods. The experimental methods include mutagenesis, X-ray crystallography, nuclear magnetic resonance (NMR) spectroscopy and Hydrogen deuterium exchange mass spectrometry (HX-MS), methods that are known in the art. As each method relies on a unique principle, the description of an epitope is intimately linked to the method by which it has been determined. Thus, depending on the epitope mapping method employed, the epitope for a given Ab/Ag pair will be described differently.

The epitope for a given antibody (Ab)/antigen (Ag) pair may be described by routine methods. For example, the overall location of an epitope may be determined by assessing the ability of an antibody to bind to different fragments or variants of the antigen. The specific amino acids within the antigen that make contact with an antibody (epitope) may also be determined using routine methods. For example, the Ab and Ag molecules may be combined and the Ab/Ag complex may be crystallized. The crystal structure of the complex may be determined and used to identify specific sites of interaction between the Ab and Ag.

“Isolated nucleic acid molecule” means a DNA or RNA polynucleotide of genomic, mRNA, cDNA, or synthetic origin or some combination thereof which is not associated with all or a portion of a polynucleotide in which the isolated polynucleotide is found in nature, or is linked to a polynucleotide to which it is not linked in nature. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences or non-coding sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

The term “PD-1 or PD-L1 binding protein” also referred to herein as a “PD-1 or PD-L1 binder” refers to an antibody, an antibody fragment, an immunoglobulin single variable domain (also referred to as “ISV” or ISVD”) or single domain antibody (also referred to as “sdAb”) that binds to PD-1 or PD-L1.

The term “immunoglobulin single variable domain” (also referred to as “ISV” or ISVD”) is generally used to refer to immunoglobulin variable domains (which may be heavy chain or light chain domains, including VH, VHH or VL domains) that can form a functional antigen-binding site without interaction with another variable domain (e.g. without a VH/VL interaction as is required between the VH and VL domains of conventional 4-chain monoclonal antibody). Examples of ISVDs will be clear to the skilled person and for example include a VHH, a humanized VHH and/or a camelized VHs such as camelized human VHs), IgNAR, domains, (single domain) antibodies (such as dAbs™) that are VH domains or that are derived from a VH domain and (single domain) antibodies (such as dAbs™) that are VL domains or that are derived from a VL domain. ISVDs that are based on and/or derived from heavy chain variable domains (such as VH or VHH domains) are generally preferred. For example, F023700906 is an ISVD.

The term “VHH” as used herein indicates that the heavy chain variable domain is obtained from or originated or derived from a heavy chain antibody. Heavy chain antibodies are functional antibodies that have two heavy chains and no light chains. Heavy chain antibodies exist in and are obtainable from Camelids (e.g., camels and alpacas), members of the biological family Camelidae. VHH antibodies have originally been described as the antigen binding immunoglobulin (variable) domain of “heavy chain antibodies” (i.e., of “antibodies devoid of light chains”; Hamers-Casterman et al., Nature 363: 446-448 (1993). The term “VHH domain” has been chosen in order to distinguish these variable domains from the heavy chain variable domains that are present in conventional four-chain antibodies (which are referred to herein as “VH domains” or “VH”) and from the light chain variable domains that are present in conventional four-chain antibodies (which are referred to herein as “VL domains” or “VL”). For a further description of VHHs, reference is made to the review article by Muyldermans (Reviews in Molec. Biotechnol. 74: 277-302, (2001), as well as to the following patent applications, which are mentioned as general background art: WO9404678, WO9504079 and WO9634103 of the Vrije Universiteit Brussel; WO9425591, WO9937681, WO0040968, WO0043507, WO0065057, WO0140310, WO0144301, EP1134231 and WO0248193 of Unilever; WO9749805, WO0121817, WO03035694, WO03054016 and WO03055527 of the Vlaams Instituut voor Biotechnologie (VI B); WO03050531 of Algonomics N.V. and Ablynx N.V.: WO0190190 by the National Research Council of Canada; WO03025020 (=EP 1433793) by the Institute of Antibodies; as well as WO2004041867, WO2004041862, WO2004041865, WO2004041863, WO2004062551, WO2005044858, WO200640153, WO2006079372, WO2006122786, WO 06122787, WO2006122825, WO2008101985, WO2008142164, and WO2015173325 by Ablynx N.V. and the further published patent applications by Ablynx N.V. Reference is also made to the further prior art mentioned in these applications, and in particular to the list of references mentioned on pages 41-43 of the International application WO 06040153, which list and references are incorporated herein by reference.

Protein electron transfer reactions play crucial roles in biological and bioenergetic redox processes. Among the amino acids involved, tyrosine serves as an efficient electron transfer intermediate. Indeed, the intrinsic reactivity of tyrosyl radicals generated from tyrosine oxidation has been effectively harnessed in peroxidase-based proximity labeling technology, where tyramide is enzymatically oxidized by heme-based peroxidases to promote labeling of proteins in proximity to the protein of interest. The reactivity of the radical is an important factor controlling the labeling radius. However, the extremely rapid phenoxy radical generation by the peroxidase enzyme limits its utility in tagging interacting cells in a spatiotemporal-controlled manner.

The present invention provides a method for highly selective synaptic tagging within a two-cell synapse using a VHH-based conjugate. This selective biotinylation approach provides the ability to specifically label and isolate uniquely interacting cells from a cell suspension to profile gene expression level differences.

The present invention provides a binding protein comprising a heavy chain antibody variable domain (VHH) that specifically binds to PD-1 or PD-L1, wherein the VHH comprises three complementarity determining regions (CDRs) selected from the group consisting of: a) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3); b) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6); c) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9); d) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and e) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15). In some embodiments, CDR1 is defined by Chothia, CDR2 is defined by Kabat, and CDR3 is defined by Kabat numbering system.

In some embodiments, the present invention provides a heavy chain antibody variable domain (VHH) that specifically binds to PD-1 or PD-L1, comprising (a) a heavy chain variable region having at least 90%, 91%, 92%, 93%, 94%, 95%, 96%, 97%, 98%, or 99% amino acid sequence identity to the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16); (b) a heavy chain variable region having at least 90%, 91%, 92%, 93%, 94%, 95%, 96%, 97%, 98%, or 99% amino acid sequence identity to the amino acid sequence: EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17); (c) a heavy chain variable region having at least 90%, 91%, 92%, 93%, 94%, 95%, 96%, 97%, 98%, or 99% amino acid sequence identity the amino acid sequence: EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or (d) a heavy chain variable region having at least 90%, 91%, 92%, 93%, 94%, 95%, 96%, 97%, 98%, or 99% amino acid sequence identity to the amino acid sequence: EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

In some embodiments, a binding protein (or “PD-1/PD-L1 binder”) is provided comprising a heavy chain antibody variable domain (VHH) that specifically binds to PD-1 or PD-L1, comprising: a) the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16); b) the amino acid sequence EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17); c) the amino acid sequence EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or d) the amino acid sequence EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

In another embodiment, a VHH-Fc fusion protein is provided comprising the amino acid sequence:

a)
(SEQ ID NO: 20)
MGWSCIILFLVATATGVHSEVQLVESGGGLVQAGGSLSVSCAPSG
RTFSNYAMGWFRQAPGKEREFVARISGGGGYTAYADSVKGRFTIA
RDNAKNTVYLQMNSLKPEDTAVYYCAAGSIDSRQPYDSTRRYDYW
GQGTLVTVSSAAADKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKASGGCLSYDTEVLTVEYGFVPIGEIVDKGI
ECSVFSIDSNGIVYTQPIAQWHHRGKQEVFEYCLEDGSIIKATKD
HKFMTQDGKMLPIDEIFEQELDLLQVKGLPEGHHHHHHG;
b)
(SEQ ID NO: 21)
MGWSCIILFLVATATGVHSEVQLVESGGGLKQAGGSLRLSCTASA
RASTYIAMAWFRRTPGKAREFVAAITWSGGHTTYADSMKGRFTIS
RDNAKNTVYLHLNALQPEDAGVYYCAANQRNTVGPSEGAYPYWGQ
GTLVTVSSAAADKTHTCPPCPAPELLGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPAPIEKTISKAKGQPR
EPQVYTLPPSREEMTKNQVSLTCLVKGFYPSDIAVEWESNGQPEN
NYKTTPPVLDSDGSFFLYSKLTVDKSRWQQGNVFSCSVMHEALHN
HYTQKSLSLSPGKASGGCLSYDTEVLTVEYGFVPIGEIVDKGIEC
SVFSIDSNGIVYTQPIAQWHHRGKQEVFEYCLEDGSIIKATKDHK
FMTQDGKMLPIDEIFEQELDLLQVKGLPEGHHHHHH
G;
c)
(SEQ ID NO: 22)
MGWSCIILFLVATATGVHSEVQLVESGGGLVQPGGSLRLSCAASG
RTLSNHAMHWFRQAPGKEREFVSAITWSDGETYYEDSVKGRFTIS
RDNAKDTAYLEMQSLKPEDTAVYYCAAKMGGPTSIPGLVEYWGQG
TLVTVSSAAADKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSREEMTKNQVSLTCLVKGFYPSDIAVEWESNGQPENN
YKTTPPVLDSDGSFFLYSKLTVDKSRWQQGNVFSCSVMHEALHNH
YTQKSLSLSPGKASGGCLSYDTEVLTVEYGFVPIGEIVDKGIECS
VFSIDSNGIVYTQPIAQWHHRGKQEVFEYCLEDGSIIKATKDHKF
MTQDGKMLPIDEIFEQELDLLQVKGLPEGHHHHHHG;
or
d)
(SEQ ID NO: 23)
MGWSCIILFLVATATGVHSEVQLVESGGGLVQAGDSLRLSCVASG
RTFSSYHMGWFRQAPGKEREFVAAIPRSGSNIGYSAFVKDRGTIS
RDNAKNTVYLQINNLAPDDTAVYYCAAKSAAGYYSGVVFTADYDY
TYWGQGTLVTVSSAAADKTHTCPPCPAPELLGGPSVFLFPPKPKD
TLMISRTPEVTCVVVDVSHEDPEVKFNWYVDGVEVHNAKTKPREE
QYNSTYRVVSVLTVLHQDWLNGKEYKCKVSNKALPAPIEKTISKA
KGQPREPQVYTLPPSREEMTKNQVSLTCLVKGFYPSDIAVEWESN
GQPENNYKTTPPVLDSDGSFFLYSKLTVDKSRWQQGNVFSCSVMH
EALHNHYTQKSLSLSPGKASGGCLSYDTEVLTVEYGFVPIGEIVD
KGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, the binding protein also includes a signal peptide. In some embodiments, the signal peptide is fused to the c-terminus of the binding protein. In some embodiments, the signal peptide is fused to the N-terminus of the binding protein. In some embodiments, the signal protein is may include the amino acid sequence:

(SEQ ID NO: 24)
MGWSCIILFLVATATGVHS.

In some embodiments, the binding protein also includes an Fc-domain. In some embodiments, the Fc-domain is fused to the N-terminus of the binding protein. In some embodiments, the Fc-domain is fused to the C-terminus of the binding protein. In some embodiments, the Fc-domain may include the amino acid sequence

(SEQ ID NO: 25)
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GKASGG.

In some embodiments, the binding protein also includes an intein. In some embodiments, the intein is fused to the C-terminus of the binding protein. In some embodiments, the intein is fused to the C-terminus of the Fc-domain. In some embodiments, the intein is fused to the N-terminus of the binding protein. In some embodiments, the intein is fused to the N-terminus of the Fc-domain. In some embodiments, the intein may include the amino acid sequence:

(SEQ ID NO: 26)
CLSYDTEVLTVEYGFVPIGEIVDKGIECSVFSIDSNGIVYTQPIA
QWHHRGKQEVFEYCLEDGSIIKATKDHKFMTQDGKMLPIDEIFEQ
ELDLLQVKGLPE.

In some embodiments, the binding protein includes a histidine tag. In some embodiments, the histidine tag is fused to the C-terminus of the binding protein. In some embodiments, the histidine tag is fused to the N-terminus of the binding protein. In some embodiments, the histidine tag is fused to the C-terminus of the Fc fusion. In some embodiments, the histidine tag is fused to the N-terminus of the Fc fusion. In some embodiments, the histidine tag is fused to the C-terminus of the intein. In some embodiments, the histidine tag is fused to the N-terminus of the intein. In some embodiments, the histidine tag includes the amino acid sequence GHHHHHHG (SEQ ID NO: 27).

In some embodiments, a VHH-Fc fusion protein is provided comprising the amino acid sequence:

(SEQ ID NO: 20)
MGWSCIILFLVATATGVHSEVQLVESGGGLVQAGGSLSVSCAPSGRTFSN
YAMGWFRQAPGKEREFVARISGGGGYTAYADSVKGRFTIARDNAKNTVYL
QMNSLKPEDTAVYYCAAGSIDSRQPYDSTRRYDYWGQGTLVTVSSAAADK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGKASGGCLSYDTEVLTVEYGFVPIGEI
VDKGIECSVFSIDSNGIVYTQPIAQWHHRGKQEVFEYCLEDGSIIKATKD
HKFMTQDGKMLPIDEIFEQELDLLQVKGLPEGHHHHHHG;

In some embodiments, an ISVD-Fc fusion protein is provided comprising the amino acid sequence:

(SEQ ID NO: 21)
MGWSCIILFLVATATGVHSEVQLVESGGGLKQAGGSLRLSCTASARASTY
IAMAWFRRTPGKAREFVAAITWSGGHTTYADSMKGRFTISRDNAKNTVYL
HLNALQPEDAGVYYCAANQRNTVGPSEGAYPYWGQGTLVTVSSAAADKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFS
CSVMHEALHNHYTQKSLSLSPGKASGGCLSYDTEVLTVEYGFVPIGEIVD
KGIECSVFSIDSNGIVYTQPIAQWHHRGKQEVFEYCLEDGSIIKATKDHK
FMTQDGKMLPIDEIFEQELDLLQVKGLPEGHHHHHHG.

In some embodiments, a VHH-Fc fusion protein is provided comprising the amino acid sequence:

(SEQ ID NO: 22)
MGWSCIILFLVATATGVHSEVQLVESGGGLVQPGGSLRLSCAASGRTLSN
HAMHWFRQAPGKEREFVSAITWSDGETYYEDSVKGRFTISRDNAKDTAYL
EMQSLKPEDTAVYYCAAKMGGPTSIPGLVEYWGQGTLVTVSSAAADKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKASGGCLSYDTEVLTVEYGFVPIGEIVDK
GIECSVFSIDSNGIVYTQPIAQWHHRGKQEVFEYCLEDGSIIKATKDHKF
MTQDGKMLPIDEIFEQELDLLQVKGLPEGHHHHHHG.

In some embodiments, a VHH-Fc fusion protein is provided comprising the amino acid sequence:

(SEQ ID NO: 23)
MGWSCIILFLVATATGVHSEVQLVESGGGLVQAGDSLRLSCVASGRTFSS
YHMGWFRQAPGKEREFVAAIPRSGSNIGYSAFVKDRGTISRDNAKNTVYL
QINNLAPDDTAVYYCAAKSAAGYYSGVVFTADYDYTYWGQGTLVTVSSAA
ADKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGKASGGCLSYDTEVLTVEYGFVPI
GEIVDKGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, a nucleic acid molecule is provided that includes a sequence of nucleotides that encodes the binding proteins of the present invention.

In some embodiments, a polypeptide is provided comprising the V_{H} domains of any of the binding proteins of the invention.

In some embodiments, an isolated nucleic acid encoding the V_{H} domains of any of the binding proteins or the polypeptides of the invention.

In some embodiments, an expression vector comprising the isolated nucleic acid the invention.

In some embodiments, the present invention provides a host cell comprising the expression vector of the invention.

The invention further comprises the nucleic acids encoding the PD-1 and PD-L1 binding proteins and fusion proteins thereof disclosed herein. For example, the present invention includes the nucleic acids described in Examples 1-20 and nucleic acids encoding the amino acids described in SEQ ID NO: 1-31.

In another embodiment, the invention provides an isolated nucleic acid or nucleic acids, for example DNA, encoding the PD-1 and PD-L1 binding proteins of the invention. In one embodiment, the isolated nucleic acid encodes an antibody or antigen binding fragment thereof comprising at least one antibody light chain variable (VL) domain and at least one antibody heavy chain variable (VH) domain, wherein the VH domain comprises at least at least three CDRs having a sequence selected from SEQ ID NOS:1-15 (e.g., SEQ ID NOs 1, 2, and 3; SEQ ID NOs, 4, 5, and 6; SEQ ID NOs 7, 8, and 9; SEQ ID NOs 10, 11, and 12, and SEQ ID NOs 13, 14, and 15). The invention also provides expression vectors comprising the isolated nucleic acids of the invention, wherein the nucleic acid is operably linked to control sequences that are recognized by a host cell when the host cell is transfected with the vector. Also provided are host cells comprising an expression vector of the present invention and methods for producing the antibody or antigen binding fragment thereof disclosed herein comprising culturing a host cell harboring an expression vector encoding the antibody or antigen binding fragment in culture medium, and isolating the antigen or antigen binding fragment thereof from the host cell or culture medium.

### Methods of Making the Binders of the Invention

The PD-1 and PD-L1 binders disclosed herein may be produced using any method known in the art. For example, the PD-1 and PD-L1 binders disclosed herein may be produced recombinantly. In this embodiment, nucleic acids encoding the PD-1 and PD-L1 binders of the invention may be inserted into a vector and expressed in a recombinant host cell. There are several methods by which to produce recombinant binders which are known in the art.

Mammalian cell lines available as hosts for expression of the PD-1 and PD-L1 binders disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the PD-1 and PD-L1 binders disclosed herein are introduced into host cells, the antibodies are produced by culturing the host cells for a period of time sufficient to allow for expression of the antibody in the host cells or, more preferably, secretion of the antibody into the culture medium in which the host cells are grown.

Proteins can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions.

The present invention further includes antibody fragments of the anti-PD-1 and anti-PD-L1 antibodies disclosed herein. The antibody fragments include F(ab)₂ fragments, which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located. An Fv fragment is a V_{L} or V_{H} region.

Immunoglobulins may be assigned to different classes depending on the amino acid sequences of the constant domain of their heavy chains. There are at least five major classes of immunoglobulins: IgA, IgD, IgE, IgG and IgM, and several of these may be further divided into subclasses (isotypes), e.g., IgG-1, IgG-2, IgG-3 and IgG-4; IgA-1 and IgA-2. The invention comprises antibodies and antigen binding fragments of any of these classes or subclasses of antibodies.

In one embodiment, the antibody or antigen binding fragment comprises a heavy chain constant region, e.g., a human constant region, such as γ1, γ2, γ3, or γ4 human heavy chain constant region or a variant thereof. In another embodiment, the antibody or antigen binding fragment comprises a light chain constant region, e.g., a human light chain constant region, such as lambda or kappa human light chain region or variant thereof. By way of example, and not limitation the human heavy chain constant region can be γ1 and the human light chain constant region can be kappa. In an alternative embodiment, the Fc region of the antibody is γ4 with a Ser228Pro mutation (Schuurman, J et. al., Mol. Immunol. 38: 1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized.

Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used.

In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Immunol. 30:105. In other instances, a modified IgG1 constant domain which has been modified to increase half-life or reduce effector function can be used.

When a term is not specifically defined herein, it has its usual meaning in the art, which will be clear to the skilled person. Reference is for example made to the standard handbooks, such as Sambrook et al., “Molecular Cloning: A Laboratory Manual” (2nd.Ed.), Vols. 1-3, Cold Spring Harbor Laboratory Press (1989); F. Ausubel et al., eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, CA (1981); Roitt et al., “Immunology” (6th. Ed.), Mosby/Elsevier, Edinburgh (2001); Roitt et al., Roitt's Essential Immunology, 10th Ed. Blackwell Publishing, UK (2001); and Janeway et al., “Immunobiology” (6th Ed.), Garland Science Publishing/Churchill Livingstone, New York (2005), as well as to the general background art cited herein.

For a general description of multivalent and multispecific polypeptides containing one or more ISVDs and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 1996/34103, WO 1999/23221, WO 2004/041862, WO 2006/122786, WO 2008/020079, WO 2008/142164 or WO 2009/068627.

The phrase “control sequences” refers to polynucleotides necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid or polynucleotide is “operably linked” when it is placed into a functional relationship with another polynucleotide. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

### Antibody Conjugates

The PD-1 and PD-L1 binder molecules disclosed herein may also be conjugated to a peptide or chemical moiety. The chemical moiety may be, inter alia, a polymer, a radionuclide or a therapeutic or cytotoxic agent. In particular embodiments, the chemical moiety is a polymer which increases the half-life of the antibody molecule in the body of a subject. Suitable polymers include, but are not limited to, hydrophilic polymers which include but are not limited to polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments disclosed herein may be pegylated, for example to increase its biological (e.g., serum) half-life. To pegylate an antibody, the antibody, or fragment thereof, typically is reacted with a reactive form of polyethylene glycol (PEG), such as a reactive ester or aldehyde derivative of PEG, under conditions in which one or more PEG groups become attached to the antibody or antibody fragment. In particular embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See, e.g., EP 0 154 316 and EP 0 401 384.

The antibodies and antibody fragments disclosed herein may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthalaldehyde, fluorescamine, 152Eu, dansyl, umbelliferone, luciferin, luminal label, isoluminal label, an aromatic acridinium ester label, an imidazole label, an acridinium salt label, an oxalate ester label, an aequorin label, 2,3-dihydrophthalazinediones, biotin/avidin, spin labels and stable free radicals. In particular embodiments, the antibodies and antibody fragments disclosed herein may also be conjugated with labels such as 99Tc, 90Y, 111In, 32P, 14C, 125I, 3H, 131I, 11C, 15O, 13N, 18F, 35S, 51Cr, 57To, 226Ra, 60Co, 59Fe, 57Se, 152Eu, 67CU, 217Ci, 211At, 212Pb, 47Sc, 109Pd, 234Th, and 40K, 157Gd, 55Mn, 52Tr, and 56Fe. Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889) disclose conjugation of various labels to antibodies.

The antibody molecules or antibody fragments may be conjugated to a cytotoxic factor such as diphtheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibody molecules to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In some embodiments, a method of producing a binding protein is provided comprising:
(a) culturing the host cell in culture medium under conditions wherein the nucleic acid sequence is expressed, thereby producing a polypeptide comprising the light and heavy chain variable regions; and
(b) recovering the polypeptides from the host cell or culture medium.

In some embodiments, a method of preparing a VHH conjugate comprising the steps of:
a) providing a VHH;
b) optionally, providing a fusion protein;
c) providing an intein; and
d) fusing the intein to the C-terminus of the VHH to form a VHH-intein conjugate or, optionally, fusing the intein to the C-terminus of the fusion protein to form a VHH-fusion protein-intein conjugate. In some embodiments, the VHH comprises three complementarity determining regions (CDRs) selected from the group consisting of: a) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3); b) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6); c) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9); d) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and e) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15). In some embodiments, the VHH comprises: (a) a heavy chain variable region having at least 90% amino acid sequence identity to the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16); (b) a heavy chain variable region having at least 90% amino acid sequence identity to the amino acid sequence: EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17); (c) a heavy chain variable region having at least 90% amino acid sequence identity the amino acid sequence: EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or (d) a heavy chain variable region having at least 90% amino acid sequence identity to the amino acid sequence: EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19). In some embodiments, the VHH comprises, a) the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16); b) the amino acid sequence EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17); c) the amino acid sequence EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or d) the amino acid sequence EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19). In some embodiments, the VHH comprises:

a)
(SEQ ID NO: 20)
MGWSCIILFLVATATGVHSEVQLVESGGGLVQAGGSLSVSCAPSGRTFSN
YAMGWFRQAPGKEREFVARISGGGGYTAYADSVKGRFTIARDNAKNTVYL
QMNSLKPEDTAVYYCAAGSIDSRQPYDSTRRYDYWGQGTLVTVSSAAADK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGKASGGCLSYDTEVLTVEYGFVPIGEI
VDKGIECSVFSIDSNGIVYTQPIAQWHHRGKQEVFEYCLEDGSIIKATKD
HKFMTQDGKMLPIDEIFEQELDLLQVKGLPEGHHHHHHG;
(SEQ ID NO: 21)
MGWSCIILFLVATATGVHSEVQLVESGGGLKQAGGSLRLSCTASARASTY
IAMAWFRRTPGKAREFVAAITWSGGHTTYADSMKGRFTISRDNAKNTVYL
HLNALQPEDAGVYYCAANQRNTVGPSEGAYPYWGQGTLVTVSSAAADKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFS
CSVMHEALHNHYTQKSLSLSPGKASGGCLSYDTEVLTVEYGFVPIGEIVD
KGIECSVFSIDSNGIVYTQPIAQWHHRGKQEVFEYCLEDGSIIKATKDHK
FMTQDGKMLPIDEIFEQELDLLQVKGLPEGHHHHHHG;
(SEQ ID NO: 22)
MGWSCIILFLVATATGVHSEVQLVESGGGLVQPGGSLRLSCAASGRTLSN
HAMHWFRQAPGKEREFVSAITWSDGETYYEDSVKGRFTISRDNAKDTAYL
EMQSLKPEDTAVYYCAAKMGGPTSIPGLVEYWGQGTLVTVSSAAADKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKASGGCLSYDTEVLTVEYGFVPIGEIVDK
GIECSVFSIDSNGIVYTQPIAQWHHRGKQEVFEYCLEDGSIIKATKDHKF
MTQDGKMLPIDEIFEQELDLLQVKGLPEGHHHHHHG;
or
(SEQ ID NO: 23)
MGWSCIILFLVATATGVHSEVQLVESGGGLVQAGDSLRLSCVASGRTFSS
YHMGWFRQAPGKEREFVAAIPRSGSNIGYSAFVKDRGTISRDNAKNTVYL
QINNLAPDDTAVYYCAAKSAAGYYSGVVFTADYDYTYWGQGTLVTVSSAA
ADKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGKASGGCLSYDTEVLTVEYGFVPI
GEIVDKGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, the fusion protein is fused to the N-terminus of the binding protein. In some embodiments, the fusion protein is fused to the C-terminus of the binding protein. In some embodiments, the Fc-domain may include the amino acid sequence

(SEQ ID NO: 25)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGKASGG.

In some embodiments, the intein is fused to the C-terminus of the binding protein. In some embodiments, the intein is fused to the C-terminus of the Fc-domain. In some embodiments, the intein is fused to the N-terminus of the binding protein. In some embodiments, the intein is fused to the N-terminus of the Fc-domain. In some embodiments, the intein may include the amino acid sequence:

(SEQ ID NO: 26)
CLSYDTEVLTVEYGFVPIGEIVDKGIECSVFSIDSNGIVYTQPIAQWHHR
GKQEVFEYCLEDGSIIKATKDHKFMTQDGKMLPIDEIFEQELDLLQVKGL
PE.

In some embodiments, VHH conjugate may also include a signal peptide. In some embodiments, the signal peptide is fused to the c-terminus of the binding protein. In some embodiments, the signal peptide is fused to the N-terminus of the binding protein. In some embodiments, the signal protein is may include the amino acid sequence: MGWSCIILFLVATATGVHS (SEQ ID NO: 24).

In some embodiments, VHH conjugate may include a histidine tag. In some embodiments, the histidine tag is fused to the C-terminus of the binding protein. In some embodiments, the histidine tag is fused to the N-terminus of the binding protein. In some embodiments, the histidine tag is fused to the C-terminus of the Fc fusion. In some embodiments, the histidine tag is fused to the N-terminus of the Fc fusion. In some embodiments, the histidine tag is fused to the C-terminus of the intein. In some embodiments, the histidine tag is fused to the N-terminus of the intein. In some embodiments, the histidine tag may include the amino acid sequence GHHHHHHG (SEQ ID NO: 27).

In some embodiments, the VHH conjugate comprises the amino acid sequence:

(SEQ ID NO: 20)
MGWSCIILFLVATATGVHSEVQLVESGGGLVQAGGSLSVSCAPSGRTFSN
YAMGWFRQAPGKEREFVARISGGGGYTAYADSVKGRFTIARDNAKNTVYL
QMNSLKPEDTAVYYCAAGSIDSRQPYDSTRRYDYWGQGTLVTVSSAAADK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGKASGGCLSYDTEVLTVEYGFVPIGEI
VDKGIECSVFSIDSNGIVYTQPIAQWHHRGKQEVFEYCLEDGSIIKATKD
HKFMTQDGKMLPIDEIFEQELDLLQVKGLPEGHHHHHHG;

In some embodiments, the VHH conjugate comprises the amino acid sequence:

(SEQ ID NO: 21)
MGWSCIILFLVATATGVHSEVQLVESGGGLKQAGGSLRLSCTASARASTY
IAMAWFRRTPGKAREFVAAITWSGGHTTYADSMKGRFTISRDNAKNTVYL
HLNALQPEDAGVYYCAANQRNTVGPSEGAYPYWGQGTLVTVSSAAADKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFS
CSVMHEALHNHYTQKSLSLSPGKASGGCLSYDTEVLTVEYGFVPIGEIVD
KGIECSVFSIDSNGIVYTQPIAQWHHRGKQEVFEYCLEDGSIIKATKDHK
FMTQDGKMLPIDEIFEQELDLLQVKGLPEGHHHHHHG.

In some embodiments, VHH conjugate comprises the amino acid sequence:

(SEQ ID NO: 22)
MGWSCIILFLVATATGVHSEVQLVESGGGLVQPGGSLRLSCAASGRTLSN
HAMHWFRQAPGKEREFVSAITWSDGETYYEDSVKGRFTISRDNAKDTAYL
EMQSLKPEDTAVYYCAAKMGGPTSIPGLVEYWGQGTLVTVSSAAADKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKASGGCLSYDTEVLTVEYGFVPIGEIVDK
GIECSVFSIDSNGIVYTQPIAQWHHRGKQEVFEYCLEDGSIIKATKDHKF
MTQDGKMLPIDEIFEQELDLLQVKGLPEGHHHHHHG.

In some embodiments, VHH conjugate comprises the amino acid sequence:

(SEQ ID NO: 23)
MGWSCIILFLVATATGVHSEVQLVESGGGLVQAGDSLRLSCVASGRTFSS
YHMGWFRQAPGKEREFVAAIPRSGSNIGYSAFVKDRGTISRDNAKNTVYL
QINNLAPDDTAVYYCAAKSAAGYYSGVVFTADYDYTYWGQGTLVTVSSAA
ADKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGKASGGCLSYDTEVLTVEYGFVPI
GEIVDKGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, a method for detecting cell-cell interactions is provided, the method comprising: a) contacting a first unlabeled cell with a VHH-photocatalyst conjugate to form a first labeled cell; b) providing a second unlabeled cell; c) combining the first labeled cell and the second unlabeled cell to form a cell-cell conjugate system; d) applying visible light in the range of approximately about 400-800 nm to the cell-cell conjugate system; e) administering a labeling probe to the cell-cell conjugate system; wherein the labeling probe covalently attaches to the first labeled cell and the second unlabeled cell in the cell-cell conjugate system; and f) detecting the labeling probe in the cell-cell conjugate system, wherein detection of the labeling probe indicates a cell-cell interaction between the first labeled cell and the second unlabeled cell. In some embodiments, the VHH-photocatalyst conjugate comprises a VHH having three complementarity determining regions (CDRs) selected from the group consisting of: a) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3); b) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6); c) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9); d) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and e) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15). In some embodiments, the VHH comprises: (a) a heavy chain variable region having at least 90% amino acid sequence identity to the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16); (b) a heavy chain variable region having at least 90% amino acid sequence identity to the amino acid sequence: EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17); (c) a heavy chain variable region having at least 90% amino acid sequence identity the amino acid sequence: EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or (d) a heavy chain variable region having at least 90% amino acid sequence identity to the amino acid sequence: EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19). In some embodiments, the VHH comprises, a) the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16); b) the amino acid sequence EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17); c) the amino acid sequence EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or d) the amino acid sequence EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19). In some embodiments, the VHH comprises:

a)
(SEQ ID NO: 20)
MGWSCIILFLVATATGVHSEVQLVESGGGLVQAGGSLSVSCAPSGRTFSN
YAMGWFRQAPGKEREFVARISGGGGYTAYADSVKGRFTIARDNAKNTVYL
QMNSLKPEDTAVYYCAAGSIDSRQPYDSTRRYDYWGQGTLVTVSSAAADK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGKASGGCLSYDTEVLTVEYGFVPIGEI
VDKGIECSVFSIDSNGIVYTQPIAQWHHRGKQEVFEYCLEDGSIIKATKD
HKFMTQDGKMLPIDEIFEQELDLLQVKGLPEGHHHHHHG;
(SEQ ID NO: 21)
MGWSCIILFLVATATGVHSEVQLVESGGGLKQAGGSLRLSCTASARASTY
IAMAWFRRTPGKAREFVAAITWSGGHTTYADSMKGRFTISRDNAKNTVYL
HLNALQPEDAGVYYCAANQRNTVGPSEGAYPYWGQGTLVTVSSAAADKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFS
CSVMHEALHNHYTQKSLSLSPGKASGGCLSYDTEVLTVEYGFVPIGEIVD
KGIECSVFSIDSNGIVYTQPIAQWHHRGKQEVFEYCLEDGSIIKATKDHK
FMTQDGKMLPIDEIFEQELDLLQVKGLPEGHHHHHHG;
(SEQ ID NO: 22)
MGWSCIILFLVATATGVHSEVQLVESGGGLVQPGGSLRLSCAASGRTLSN
HAMHWFRQAPGKEREFVSAITWSDGETYYEDSVKGRFTISRDNAKDTAYL
EMQSLKPEDTAVYYCAAKMGGPTSIPGLVEYWGQGTLVTVSSAAADKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKASGGCLSYDTEVLTVEYGFVPIGEIVDK
GIECSVFSIDSNGIVYTQPIAQWHHRGKQEVFEYCLEDGSIIKATKDHKF
MTQDGKMLPIDEIFEQELDLLQVKGLPEGHHHHHHG;
or
(SEQ ID NO: 23)
MGWSCIILFLVATATGVHSEVQLVESGGGLVQAGDSLRLSCVASGRTFSS
YHMGWFRQAPGKEREFVAAIPRSGSNIGYSAFVKDRGTISRDNAKNTVYL
QINNLAPDDTAVYYCAAKSAAGYYSGVVFTADYDYTYWGQGTLVTVSSAA
ADKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGKASGGCLSYDTEVLTVEYGFVPI
GEIVDKGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, VHH-photocatalyst conjugate includes a fusion protein. In some embodiments, the fusion protein is fused to the N-terminus of the VHH. In some embodiments, the fusion protein is fused to the C-terminus of the VH. In some embodiments, the Fc-domain may include the amino acid sequence

(SEQ ID NO: 25)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGKASGG.

In some embodiments, VHH-photocatalyst conjugate may include an intein. In some embodiments, the intein is fused to the C-terminus of the binding protein. In some embodiments, the intein is fused to the C-terminus of the Fc-domain. In some embodiments, the intein is fused to the N-terminus of the binding protein. In some embodiments, the intein is fused to the N-terminus of the Fc-domain. In some embodiments, the intein may include the amino acid sequence:

(SEQ ID NO: 26)
CLSYDTEVLTVEYGFVPIGEIVDKGIECSVFSIDSNGIVYTQPIAQWHHR
GKQEVFEYCLEDGSIIKATKDHKFMTQDGKMLPIDEIFEQELDLLQVKGL
PE.

In some embodiments, VHH-photocatalyst conjugate may also include a signal peptide. In some embodiments, the signal peptide is fused to the c-terminus of the binding protein. In some embodiments, the signal peptide is fused to the N-terminus of the binding protein. In some embodiments, the signal protein is may include the amino acid sequence:

| (SEQ ID NO: 24) | |
|---|---|
| | MGWSCIILFLVATATGVHS. |

In some embodiments, VHH-photocatalyst conjugate may include a histidine tag. In some embodiments, the histidine tag is fused to the C-terminus of the binding protein. In some embodiments, the histidine tag is fused to the N-terminus of the binding protein. In some embodiments, the histidine tag is fused to the C-terminus of the Fc fusion. In some embodiments, the histidine tag is fused to the N-terminus of the Fc fusion. In some embodiments, the histidine tag is fused to the C-terminus of the intein. In some embodiments, the histidine tag is fused to the N-terminus of the intein. In some embodiments, the histidine tag may include the amino acid sequence GHHHHHHG (SEQ ID NO: 27).

In some embodiments, the VHH-photocatalyst conjugate comprises the amino acid sequence:

(SEQ ID NO: 20)
MGWSCIILFLVATATGVHSEVQLVESGGGLVQAGGSLSVSCAPSGRTFSN
YAMGWFRQAPGKEREFVARISGGGGYTAYADSVKGRFTIARDNAKNTVYL
QMNSLKPEDTAVYYCAAGSIDSRQPYDSTRRYDYWGQGTLVTVSSAAADK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGKASGGCLSYDTEVLTVEYGFVPIGEI
VDKGIECSVFSIDSNGIVYTQPIAQWHHRGKQEVFEYCLEDGSIIKATKD
HKFMTQDGKMLPIDEIFEQELDLLQVKGLPEGHHHHHHG;

In some embodiments, the VHH-photocatalyst conjugate comprises the amino acid sequence:

(SEQ ID NO: 21)
MGWSCIILFLVATATGVHSEVQLVESGGGLKQAGGSLRLSCTASARASTY
IAMAWFRRTPGKAREFVAAITWSGGHTTYADSMKGRFTISRDNAKNTVYL
HLNALQPEDAGVYYCAANQRNTVGPSEGAYPYWGQGTLVTVSSAAADKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFS
CSVMHEALHNHYTQKSLSLSPGKASGGCLSYDTEVLTVEYGFVPIGEIVD
KGIECSVFSIDSNGIVYTQPIAQWHHRGKQEVFEYCLEDGSIIKATKDHK
FMTQDGKMLPIDEIFEQELDLLQVKGLPEGHHHHHHG.

In some embodiments, VHH-photocatalyst conjugate comprises the amino acid sequence:

(SEQ ID NO: 22)
MGWSCIILFLVATATGVHSEVQLVESGGGLVQPGGSLRLSCAASGRTLSN
HAMHWFRQAPGKEREFVSAITWSDGETYYEDSVKGRFTISRDNAKDTAYL
EMQSLKPEDTAVYYCAAKMGGPTSIPGLVEYWGQGTLVTVSSAAADKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKASGGCLSYDTEVLTVEYGFVPIGEIVDK
GIECSVFSIDSNGIVYTQPIAQWHHRGKQEVFEYCLEDGSIIKATKDHKF
MTQDGKMLPIDEIFEQELDLLQVKGLPEGHHHHHHG.

In some embodiments, VHH-photocatalyst conjugate comprises the amino acid sequence:

(SEQ ID NO: 23)
MGWSCIILFLVATATGVHSEVQLVESGGGLVQAGDSLRLSCVASGRTFSS
YHMGWFRQAPGKEREFVAAIPRSGSNIGYSAFVKDRGTISRDNAKNTVYL
QINNLAPDDTAVYYCAAKSAAGYYSGVVFTADYDYTYWGQGTLVTVSSAA
ADKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGKASGGCLSYDTEVLTVEYGFVPI
GEIVDKGIECSVFSIDSNGIVYTQPIAQWHHRGKQEVFEYCLEDGSIIKA
TKDHKFMTQDGKMLPIDEIFEQELDLLQVKGLPEGHHHHHHG.

In some embodiments, the visible light activates the VHH-photocatalyst conjugate. In some embodiments, the cell-cell interaction is only observed in visible light. In some embodiments, the VHH-photocatalyst activates the labeling probe.

In some embodiments, the labeling probe is biotin tyramide.

In some embodiments, the VHH-photocatalyst conjugate does not block PD-1/PD-L1 interaction of the cell. In some embodiments, the first unlabeled cell comprises PD-L1.

In some embodiments, the first unlabeled cell comprises PD-1. In some embodiments, the second unlabeled cell comprises PD-L1. In some embodiments, the second unlabeled cell comprises PD-1.

In some embodiments, the VHH-photocatalyst conjugate does not block PD-1/PD-L1 interaction of the cell. In some embodiments, the VHH-photocatalyst conjugate binds to PD-L1 at the cell surface, but does not disrupt PD-1/PD-L1-mediated inhibition of IL-2 production. In some embodiments, the VHH-photocatalyst conjugate provides minimal disruption of the PD-1/PD-L1 binding interaction.

In some embodiments, SEQ ID NOs. 16 and 17 are an anti-PD-L1 non-blocking VHH. In some embodiments, SEQ ID NO. 18 is an anti-PD-L1 blocking VHH. In some embodiments, SEQ ID NO. 19 is an anti-PD-1 VHH with minimal PD-L1 blocking activity.

### Experimental and Diagnostic Uses

The PD-1 and PD-L1 binders disclosed herein may be used as affinity purification agents. In this process, the binders are immobilized on a solid phase such a Sephadex resin or filter paper, using methods well known in the art. The immobilized PD-1 and PD-L1 binders are contacted with a sample containing the target protein (or fragment thereof) to be purified, and thereafter the support is washed with a suitable solvent that will remove substantially all the material in the sample except the target protein, which is bound to the immobilized binder. Finally, the support is washed with a solvent which elutes the bound target from the column (e.g., a Protein A column, such as ThermoFisher's MabCapture C Protein A Chromatography Resin). Such immobilized PD-1 and PD-L1 binders form part of the present invention.

Further provided are antigens for generating secondary antibodies which are useful for example for performing Western blots and other immunoassays discussed herein. in particular, polypeptides are disclosed which comprise the variable regions and/or CDR sequences of a therapeutic antibody disclosed herein and which may be used to generate an anti-idiotypic antibodies for use in specifically detecting the presence of the antibody, e.g., in a therapeutic context.

Anti-PD-1 and PD-L1 binders or fragments thereof may also be useful in diagnostic assays for the target protein, e.g., detecting its expression in specific cells, tissues, or serum. Such diagnostic methods may be useful in various disease diagnoses.

Imaging techniques include SPECT imaging (single photon emission computed tomography) or PET imaging (positron emission tomography). Labels include e.g., iodine-123 (123I) and technetium-99m (99mTc), e.g., in conjunction with SPECT imaging or 11C, 13N, 15O or 18F, e.g., in conjunction with PET imaging or Indium-111 (See e.g., Gordon et al., (2005) International Rev. Neurobiol. 67:385-440).

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2nd ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

In embodiment 1, a binding protein is provided comprising a heavy chain antibody variable domain (VHH) that specifically binds to PD-1 or PD-L1, wherein the VHH comprises three complementarity determining regions (CDRs) selected from the group consisting of:
a) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3);
b) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6);
c) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9);
d) a CDR1 comprising the amino acid sequence GRTLSNHAMH (SEQ ID NO: 7), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and
e) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15).

In embodiment 2, the binding protein of embodiment 1 is provided wherein the VHH comprises:
(a) a heavy chain variable region having at least 90% amino acid sequence identity to the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16);
(b) a heavy chain variable region having at least 90% amino acid sequence identity to the amino acid sequence: EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17);
(c) a heavy chain variable region having at least 90% amino acid sequence identity the amino acid sequence: EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or
(d) a heavy chain variable region having at least 90% amino acid sequence identity to the amino acid sequence: EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

In embodiment 3, the binging protein of embodiment 1 is provided wherein the VHH comprises:
a) the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16);
b) the amino acid sequence EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17);
c) the amino acid sequence EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or
d) the amino acid sequence EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

In embodiment 4, the binding protein of any of embodiments 1-3 is provided, further comprising a signal peptide comprising the amino acid sequence: MGWSCIILFLVATATGVHS (SEQ ID NO: 24).

In embodiment 5, the binding protein of embodiment 4 is provided, wherein the C-terminus of the signal peptide is fused to the N-terminus of the binding protein.

In embodiment 6, the binding protein comprising the VHH of any of embodiments 1-4 fused to an Fc-domain comprising the amino acid sequence DKTHTCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTISK AKGQPREPQVYTLPPSREEMTKNQVSLTCLVKGFYPSDIAVEWESNGQPENNYKTTPPV LDSDGSFFLYSKLTVDKSRWQQGNVFSCSVMHEALHNHYTQKSLSLSPGKASGG (SEQ ID NO: 25), is provided.

In embodiment 7, the binding protein of embodiment 6 is provided, wherein the C-terminus of the Fc-domain is fused to the N-terminus of the binding protein.

In embodiment 8, the binding protein of any of embodiments 6-7 is provided, further comprising an intein comprising the amino acid sequence:

(SEQ ID NO: 26)
CLSYDTEVLTVEYGFVPIGEIVDKGIECSVFSIDSNGIVYTQPIAQWHHR
GKQEVFEYCLEDGSIIKATKDHKFMTQDGKMLPIDEIFEQELDLLQVKGL
PE.

In embodiment 9, the binding protein of embodiment 8 is provided, wherein the C-terminus of the intein is fused to the N-terminus of the binding protein.

In embodiment 10, the binding protein of any of embodiments 8-9 is provided, further comprising a histidine tag comprising the amino acid sequence GHHHHHHG (SEQ ID NO: 27).

In embodiment 11, the binding protein of embodiment 10 is provided, wherein the C-terminus of the histidine tag is fused to the N-terminus of the intein.

In embodiment 12, an VHH-Fc fusion protein is provided comprising the amino acid sequence:

(SEQ ID NO: 20)
a) MGWSCIILFLVATATGVHSEVQLVESGGGLVQAGGSLSVSCAPSGRTFSNYAM
GWFRQAPGKEREFVARISGGGGYTAYADSVKGRFTIARDNAKNTVYLQMNSLK
PEDTAVYYCAAGSIDSRQPYDSTRRYDYWGQGTLVTVSSAAADKTHTCPPCPAP
ELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGKAS
GGCLSYDTEVLTVEYGFVPIGEIVDKGIECSVFSIDSNGIVYTQPIAQWHHRGKQE
VFEYCLEDGSIIKATKDHKFMTQDGKMLPIDEIFEQELDLLQVKGLPEGHHHHHHG;
(SEQ ID NO: 21)
b) MGWSCIILFLVATATGVHSEVQLVESGGGLKQAGGSLRLSCTASARASTYIAMA
WFRRTPGKAREFVAAITWSGGHTTYADSMKGRFTISRDNAKNTVYLHLNALQP
EDAGVYYCAANQRNTVGPSEGAYPYWGQGTLVTVSSAAADKTHTCPPCPAPEL
LGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGKASG
GCLSYDTEVLTVEYGFVPIGEIVDKGIECSVFSIDSNGIVYTQPIAQWHHRGKQEV
FEYCLEDGSIIKATKDHKFMTQDGKMLPIDEIFEQELDLLQVKGLPEGHHHHHHG;
(SEQ ID NO: 22)
c) MGWSCIILFLVATATGVHSEVQLVESGGGLVQPGGSLRLSCAASGRTLSNHAMH
WFRQAPGKEREFVSAITWSDGETYYEDSVKGRFTISRDNAKDTAYLEMQSLKPE
DTAVYYCAAKMGGPTSIPGLVEYWGQGTLVTVSSAAADKTHTCPPCPAPELLG
GPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGKASGG
CLSYDTEVLTVEYGFVPIGEIVDKGIECSVFSIDSNGIVYTQPIAQWHHRGKQEVF
EYCLEDGSIIKATKDHKFMTQDGKMLPIDEIFEQELDLLQVKGLPEGHHHHHHG;
or
(SEQ ID NO: 23)
d) MGWSCIILFLVATATGVHSEVQLVESGGGLVQAGDSLRLSCVASGRTFSSYHMG
WFRQAPGKEREFVAAIPRSGSNIGYSAFVKDRGTISRDNAKNTVYLQINNLAPDD
TAVYYCAAKSAAGYYSGVVFTADYDYTYWGQGTLVTVSSAAADKTHTCPPCP
APELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPG
KASGGCLSYDTEVLTVEYGFVPIGEIVDKGIECSVFSIDSNGIVYTQPIAQWHHRG
KQEVFEYCLEDGSIIKATKDHKFMTQDGKMLPIDEIFEQELDLLQVKGLPEGHHH
HHHG.

In embodiment 13, a nucleic acid molecule is provided comprising a sequence of nucleotides that encodes the binding protein of any of embodiments 1-11 or the VHH-Fc fusion protein of embodiment 12.

In embodiment 14, a polypeptide is provided comprising the V_{H} domains of any of the binding proteins of any of embodiments 1-11.

In embodiment 15, an isolated nucleic acid is provided encoding the V_{H} domains of any of the binding proteins of any of embodiments 1-11, or the polypeptide of embodiment 14.

In embodiment 16, an expression vector is provided comprising the isolated nucleic acid of embodiment 15.

In embodiment 17, a host cell is provided comprising the expression vector of embodiment 16.

In embodiment 18, a method of producing a binding protein is provided, according to any of embodiments 1-11 comprising:
a) culturing the host cell of embodiment 17 in culture medium under conditions wherein the nucleic acid sequence is expressed, thereby producing a polypeptide comprising the light and heavy chain variable regions; and
b) recovering the polypeptides from the host cell or culture medium.

In embodiment 19, a method of preparing a VHH conjugate is provided comprising the steps of:
a) providing a VHH having the amino acid sequence of any of embodiments 1-11;
b) optionally, providing a fusion protein;
c) providing an intein; and
d) fusing the intein to the C-terminus of the VHH to form a VHH-intein conjugate or, optionally, fusing the intein to the C-terminus of the fusion protein to form a VHH-fusion protein-intein conjugate.

In embodiment 20, a method for detecting cell-cell interactions is provided, the method comprising:
a) contacting a first unlabeled cell with a VHH-photocatalyst conjugate comprising: comprises a VHH having three complementarity determining regions (CDRs) selected from the group consisting of:
   i) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3);
   ii) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6);
   iii) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9);
   iv) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and
   v) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15) to form a first labeled cell;
b) providing a second unlabeled cell;
c) combining the first labeled cell and the second unlabeled cell to form a cell-cell conjugate system;
d) applying visible light in the range of approximately about 400-800 nm to the cell-cell conjugate system;
e) administering a labeling probe to the cell-cell conjugate system;
wherein the labeling probe covalently attaches to the first labeled cell and the second unlabeled cell; and
f) observing the labeling probe results in detection of the cell-cell interaction.

In embodiment 21, the method of embodiment 20 is provided, wherein applying visible light activates the VHH-photocatalyst.

In embodiment 22, the method of any of embodiments 20-21 is provided, wherein the interaction is only observed in visible light.

In embodiment 23, the method of any of embodiments 20-22 is provided, wherein the VHH-photocatalyst activates the labeling probe.

In embodiment 24, the method of any of embodiments 20-23 is provided, wherein the labeling probe is biotin tyramide.

In embodiment 25, the method of any of embodiments 20-24 is provided, wherein the first unlabeled cell comprises PD-L1.

In embodiment 26, the method of any of embodiments 20-25 is provided, wherein the first unlabeled cell comprises PD-1.

In embodiment 27, the method of any of embodiments 20-26 is provided, wherein the second unlabeled cell comprises PD-L1.

In embodiment 28, the method of any of embodiments 20-27 is provided, wherein the second unlabeled cell comprises PD-1.

In embodiment 29, the method of any of embodiments 20-28 is provided, wherein the VHH-photocatalyst conjugate does not block PD-1/PD-L1 interaction of the cell.

In embodiment 30, the method of any of embodiments 20-29 is provided, wherein the VHH-photocatalyst conjugate binds to PD-L1 at the cell surface, but does not disrupt PD-1/PD-L1-mediated inhibition of IL-2 production.

In embodiment 31, the method of any of embodiments 20-30 is provided, wherein the VHH-photocatalyst conjugate provides minimal disruption of the PD-1/PD-L1 binding interaction.

### Examples

These examples are intended to exemplify the present invention are not a limitation thereof. Compositions and methods set forth in the Examples form part of the present invention.

### Example 1: Single Domain Antibody (VHH) Generation

An alpaca was immunized with a DNA expression vector encoding human PD-L1 and PD-1. Blood samples were collected and peripheral blood mononuclear cells prepared using Ficoll-Hypaque according to the manufacturer's instructions (Amersham Biosciences). Total RNA was extracted from the cells and used as starting material for RT-PCR to amplify the VHH-encoding DNA segments. These segments were then cloned into a phage display vector to create VHH antibody libraries, as described in WO 05/044858, the contents of which are incorporated herein by reference. The phage were prepared according to standard protocols and stored after filter sterilization at 4° C. for further use. Biotinylated human PD-L1 and PD-1 was used for two rounds of in-solution selection of the VHH library, selected phage were plated, and individual phage were transferred to a 96 well plate.

Phage-encoded VHH antibodies were expressed in the E. coli expression vectors and periplasmic extracts were prepared. Primary screening focused on flow cytometry-based binding to CHO cells expressing human PD-L1 (CHO-K1.hPD-L1) or human PD-1 (CHO-K1.hPD-1) human PD-L2 or surface plasmon resonance-based binding. Secondary screening indicated the presence of PD-L1 binding and/or PD-1-non-blocking/CD80-non-blocking VHHs or PD-1 binding VHHs with acceptable off-rates that could be used as a proximity labeling tool. Selected non-blocking or blocking PD-L1 VHHs and PD-1 VHHs were recloned into the E. coli expression vector with a C-terminal FLAG3-HIS6 tag. Electrocompetent cells were transformed, positive clones were sequenced, shake flask cultures started, and VHH purified via IMAC purification.

### Example 2: Binding to Human PD-L1

CHO-K1.hPD-L1 cells were resuspended in Stain Buffer (PBS, 2% FBS, 0.05% sodium azide) and 1E5 cells/well were transferred to a 96-well V-bottom plate and centrifuged. Cells were suspended in 100 μL/well serial diluted VHH or anti-PD-L1 antibody (atezolizumab) in Stain Buffer and incubated for 30 minutes at 4° C. VHH binding was detected by resuspending the samples subsequently in 100 μL/well mouse a-FLAG antibody (Sigma: F1804) and detected with PE-labeled goat anti-mouse IgG (Jackson ImmunoResearch: 115-116-071) as detection antibody and 5 nM TOPRO3 (Molecular Probes: T3605) as dead dye. Between each step, the cells were collected via centrifugation for 5 minutes at 200×g and washed three times with 100 μL/well Stain Buffer.

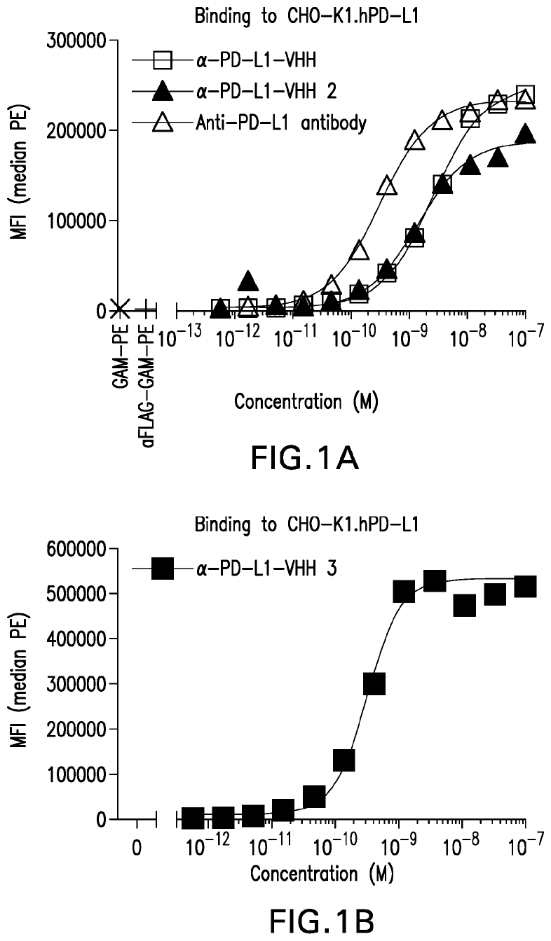

FIG. 1A represents the binding of α-PD-L1 VHH and α-PD-L1 VHH2 to CHO-K1.hPD-L1 expression cells and FIG. 1B represents the binding of α-PD-L1 VHH3 to CHO-K1.hPD-L1 expression cells. As shown in FIGS. 1A and 1B, all three VHHs indicated low nanomolar affinity for PD-L1.

### Example 3: Blockade of Human PD-1 Binding to Human PD-L1

CHO-K1.hPD-L1 cells were resuspended in Stain Buffer (PBS, 2% FBS, 0.05% sodium azide) and 1E5 cells/well were transferred to 96-well V-bottom plates and centrifuged. Cells were suspended in 100 μL mixture of serially diluted VHH or anti-PD-L1 antibody (atezolizumab) and 9.3 nM human PD-1-Fc in Stain Buffer and incubated for 1.5 hours at 4° C., 300 rpm. Residual binding of human PD-L1-Fc was detected with 100 μL PE-labeled goat anti-human IgG antibody (Southern Biotech: 2043 09). Between each step, the cells were collected via centrifugation for 5 minutes at 200×g and washed three times with 100 μL/well Stain Buffer. Prior to analysis, the samples were resuspended in 5 nM TOPRO3 (Molecular Probes: T3605) to exclude dead cells.

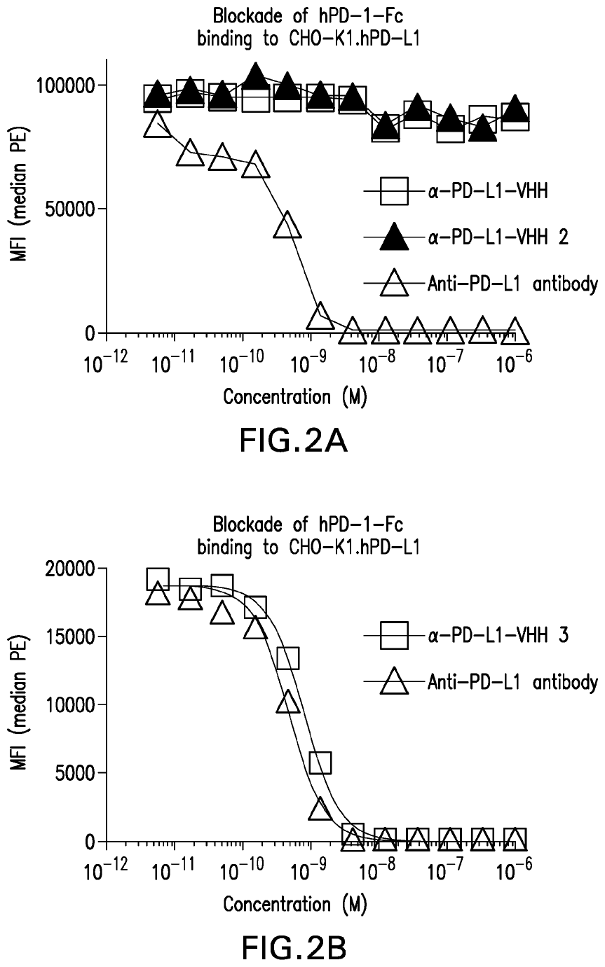

FIG. 2A represent PD-1-Fc mediated blockade of α-PD-L1 VHH, α-PD-L1 VHH2, and 2B represents α-PD-L1 VHH 3 binding to CHO-K1.hPD-L1 expression cells. As shown in FIG. 2A, α-PD-L1 VHH and α-PD-L1 VHH 2 do not block binding between PD-1 and PD-L1 while α-PD-L1 VHH 3 shows strong blocking effects similar to an anti-PD-L1 blocking antibody, as shown in FIG. 2B.

### Example 4: Blockade of Human CD80 Binding to Human PD-L1

An ELISA was performed in a final volume of 25 μL in 384HB Spectraplates (PerkinElmer: 6007500). 2 μg/mL human CD80-Fc (Sino Biological: 10698-H03H) was coated overnight at 4° C. Assay plates were blocked with a 1% casein solution in PBS for at least 1 hour at room temperature. A serial dilution of anti-PD-L1 VHH or anti-PD-L1 antibody (atezolizumab) was prepared in assay buffer (PBS, 0.05% Tween 20, 0.1% casein) and mixed with an equal volume of biotinylated hPD-L1-Fc diluted in assay buffer to obtain a final concentration of 50 nM. The samples were added to the appropriate wells and after 1-hour incubation at room temperature, residual binding of biotinylated hPD-L1-Fc was detected with extravidin-HRP (Sigma:E2886). Absorbance at 450 nm was measured with the Tecan Infinite (Tecan) after addition of esTMB substrate (SDT GmbH:esTMB) and HCL (1M).

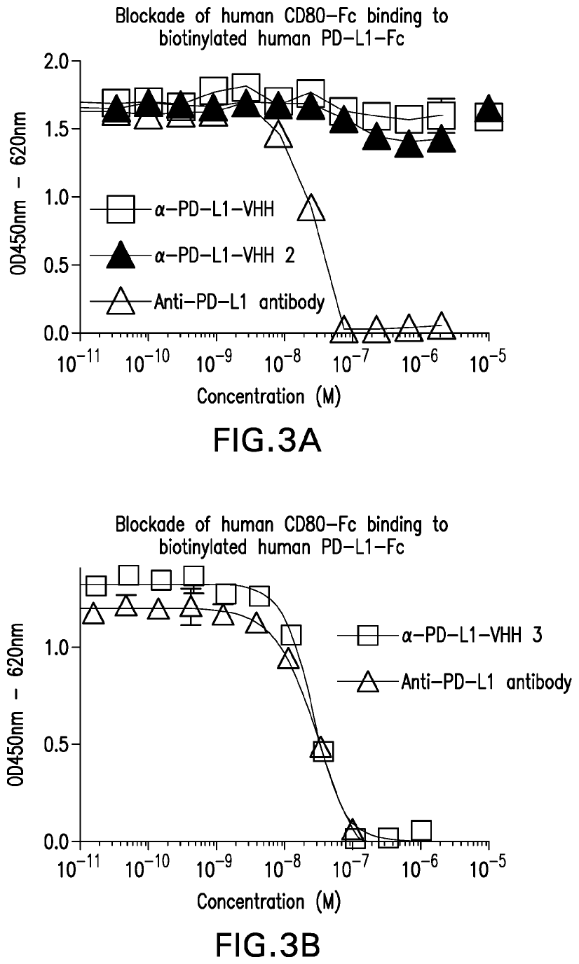

FIG. 3A represent α-PD-L1 VHH, α-PD-L1 VHH 2, and 3B represents α-PD-L1 VHH 3 mediated blockade of human CD80-Fc binding to biotinylated human PD-L1-Fc.

### Example 5: Binding to Human PD-1

CHO-K1.hPD-1 cells were resuspended in Stain Buffer (PBS, 2% FBS, 0.05% sodium azide) and 1E5 cells/well were transferred to a 96-well V-bottom plate and centrifuged. Cells were suspended in 100 μl/well serial diluted VHH or antibody in Stain Buffer and incubated for 30 minutes at 4° C. VHH binding was detected by resuspending the samples subsequently in 100 μl/well mouse a-FLAG antibody (Sigma: F1804) and detected with PE-labeled goat anti-mouse IgG (Jackson ImmunoResearch: 115-116-071) as detection antibody and 5 nM TOPRO3 (Molecular Probes: T3605) as dead dye. Between each step, the cells were collected via centrifugation for 5 minutes at 200×g and washed three times with 100 μl/well Stain Buffer.

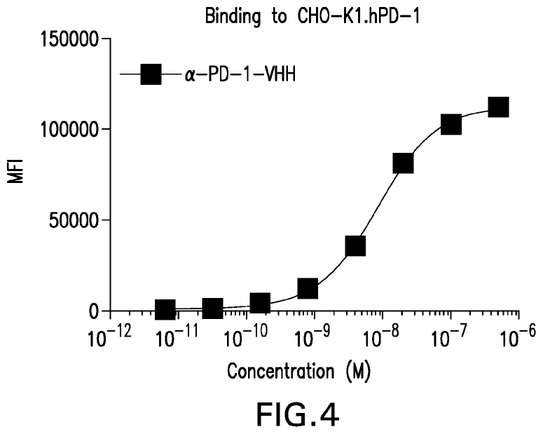

FIG. 4 represents the binding of α-PD-1 VHH to CHO-K1.hPD-1 expression cells.

### Example 6: Blockade of Human PD-L1 Binding to Human PD-1

CHO-K1.hPD-1 cells were resuspended in Stain Buffer (PBS, 2% FBS, 0.05% sodium azide) and 1E5 cells/well were transferred to 96-well V-bottom plates and centrifuged. Cells were suspended in 100 μl mixture of serially diluted VHH or anti-PD-1 blocking antibody and 9.3 nM human PD-L1-Fc in Stain Buffer and incubated for 1.5 hours at 4° C., 300 rpm. Residual binding of human PD-L1-Fc was detected with 100 μl PE-labeled goat anti-human IgG antibody (Southern Biotech: 2043 09). Between each step, the cells were collected via centrifugation for 5 minutes at 200×g and washed three times with 100 μl/well Stain Buffer. Prior to analysis, the samples were resuspended in 5 nM TOPRO3 (Molecular Probes: T3605) to exclude dead cells.

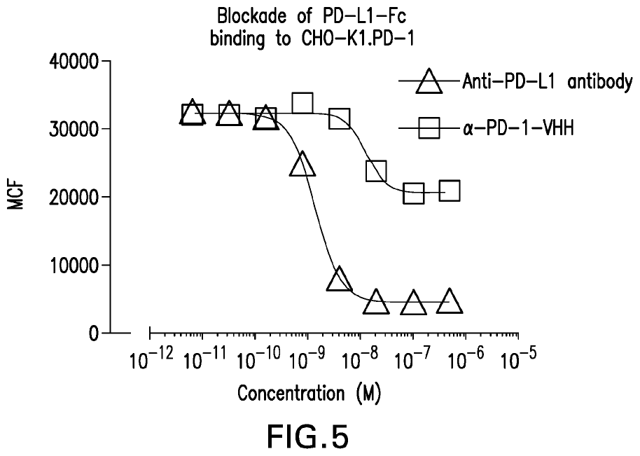

FIG. 5 represents α-PD-1 VHH mediated blockade of PD-L1-Fc binding to CHO-K1.hPD-L1 expression cells. As shown in FIG. 5, α-PD-1 VHH shows partial blocking of the PD-L1 and PD-1 interaction compared to an anti-PD-1 blocking antibody.

### Example 7: Preparation of VHH Fc-N3 Conjugates using F0237634F06 (Cloning of isotype, PD-L1, and PD-1 VHHs)

The DNA encoding the isotype VHH, α-PD-L1 VHH (non-blocking), α-PD-L1 2 VHH (non-blocking), α-PD-L1 3 VHH (blocking), and α-PD-1 VHH (non-blocking) including an N-terminal signal peptide, were purchased from Genewiz (Germany) and fused to the N-terminus of the CH2 domain of the previously described pFUSEN-hG1Fc-IntN15 vector via overlap extension PCR¹⁶ to obtain the construction Isotype-VHH-Fc-AvaN and PD-L1-VHH-FC-AvaN. The resulting plasmids encoded for the following PD-L1 and PD-1 VHH sequences:

α-PD-L1 VHH-Fc sequence:
(SEQ ID NO: 20)
MGWSCIILFLVATATGVHSEVQLVESGGGLVQAGGSLSVSCAPSGRTFSNYAMGWFRQ
APGKEREFVARISGGGGYTAYADSVKGRFTIARDNAKNTVYLQMNSLKPEDTAVYYCA
AGSIDSRQPYDSTRRYDYWGQGTLVTVSSAAADKTHTCPPCPAPELLGGPSVFLFPPKPK
DTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFS
CSVMHEALHNHYTQKSLSLSPGKASGGCLSYDTEVLTVEYGFVPIGEIVDKGIECSVFSID
SNGIVYTQPIAQWHHRGKQEVFEYCLEDGSIIKATKDHKFMTQDGKMLPIDEIFEQELDL
LQVKGLPEGHHHHHHG
α-PD-L1 VHH 2-Fc sequence:
(SEQ ID NO: 21)
MGWSCIILFLVATATGVHSEVQLVESGGGLKQAGGSLRLSCTASARASTYIAMAWFRRT
PGKAREFVAAITWSGGHTTYADSMKGRFTISRDNAKNTVYLHLNALQPEDAGVYYCAA
NQRNTVGPSEGAYPYWGQGTLVTVSSAAADKTHTCPPCPAPELLGGPSVFLFPPKPKDT
LMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCS
VMHEALHNHYTQKSLSLSPGKASGGCLSYDTEVLTVEYGFVPIGEIVDKGIECSVFSIDS
NGIVYTQPIAQWHHRGKQEVFEYCLEDGSIIKATKDHKFMTQDGKMLPIDEIFEQELDLL
QVKGLPEGHHHHHHG
α-PD-L1 VHH 3-Fc sequence:
(SEQ ID NO: 22)
MGWSCIILFLVATATGVHSEVQLVESGGGLVQPGGSLRLSCAASGRTLSNHAMHWFRQ
APGKEREFVSAITWSDGETYYEDSVKGRFTISRDNAKDTAYLEMQSLKPEDTAVYYCAA
KMGGPTSIPGLVEYWGQGTLVTVSSAAADKTHTCPPCPAPELLGGPSVFLFPPKPKDTL
MISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSV
MHEALHNHYTQKSLSLSPGKASGGCLSYDTEVLTVEYGFVPIGEIVDKGIECSVFSIDSN
GIVYTQPIAQWHHRGKQEVFEYCLEDGSIIKATKDHKFMTQDGKMLPIDEIFEQELDLLQ
VKGLPEGHHHHHHG
α-PD-1 VHH-Fc sequence:
(SEQ ID NO: 23)
MGWSCIILFLVATATGVHSEVQLVESGGGLVQAGDSLRLSCVASGRTFSSYHMGWFRQ
APGKEREFVAAIPRSGSNIGYSAFVKDRGTISRDNAKNTVYLQINNLAPDDTAVYYCAA
KSAAGYYSGVVFTADYDYTYWGQGTLVTVSSAAADKTHTCPPCPAPELLGGPSVFLFPP
KPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGN
VFSCSVMHEALHNHYTQKSLSLSPGKASGGCLSYDTEVLTVEYGFVPIGEIVDKGIECSV
FSIDSNGIVYTQPIAQWHHRGKQEVFEYCLEDGSIIKATKDHKFMTQDGKMLPIDEIFEQE
LDLLQVKGLPEGHHHHHHG

### Example 8: Expression and purification of Isotype and PD-L1 VHHs

Expi293 cells were transiently transfected with either Isotype-VHH-Fc-AvaN or PD-L1-VHH-FC-AvaN constructs using Expifectamine (Life Technology), according to the manufacturer's instructions.

In all cases, after 6 days incubation at 37° C. with 8% CO₂, cell supernatants were harvested and spun down at 2000 rcf for 20 min at 4° C. After addition of complete protease inhibitors cell supernatants were dialyzed against PBS and directly purified over a Protein A column. For Western blot analysis, samples were loaded onto 8% acrylamide Bis-Tris gels and run in MES-SDS running buffer. Pure fractions were pooled and stored at −80° C.

### Example 9: Generation of NpuC-Cys-OMe (IntC) Protein and Cys-2N3-Linker

NpuC-Cys-OMe was obtained via thiolysis from a Mxe GyrA intein fusion The linker was custom-made at the Pompeu Fabra University Peptide Synthesis Facility (Barcelona, Spain) by standard Fmoc SPPS (solid phase peptide synthesis) on 2-Chlorotrityl resin (0.25 mmol/g, Iris Biotech, Marktredwitz Deustchland). Chain assembly was carried out with HBTU activation (4.8 eq) using a 5-fold excess of amino acid over the resin in DMF (dimethylformamide) with DIEA (N,N-diisopropylethylamine). The Fmoc protecting group was removed with 20% piperidine in DMF (1×2 minutes, followed by 2×10 minutes). Peptidyl-resin was washed between coupling cycles with DMF for 3 minutes by alternating batch or flow washes. Peptide was cleaved from the resin using 94% TFA, 1% triisopropylsilane (TIS), 2.5% ethanedithiol, and 2.5% H2O (cleavage cocktail). Crude peptide products were precipitated and washed with cold Et2O, dissolved in solvent A (0.1% TFA in water) with a minimal amount of solvent B (0.1% TFA 90% acetonitrile in water) and then purified by RP-HPLC.

Chemical structure of Cys-2N3 linker compound (Cys-2N3) is represented by the structure of Formula I below:

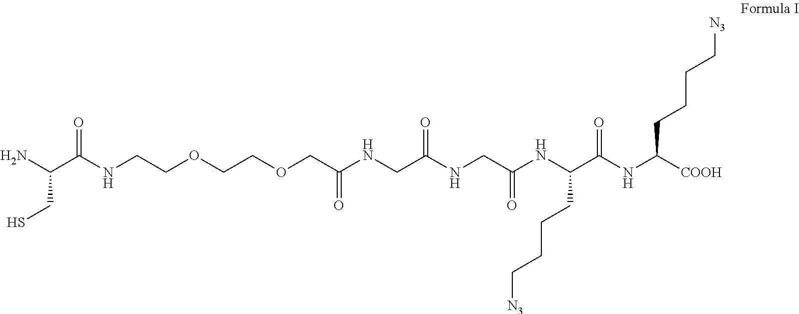 [SMILES: [N-]=[N+]=NCCCC[C@H](NC(=O)[C@H](CCCCN=[N+]=[N-])NC(=O)CNC(=O)CNC(=O)COCCOCCNC(=O)[C@@H](N)CS)C(=O)O]

### Example 10: SEPL Reaction of Cys-2N3-Linker

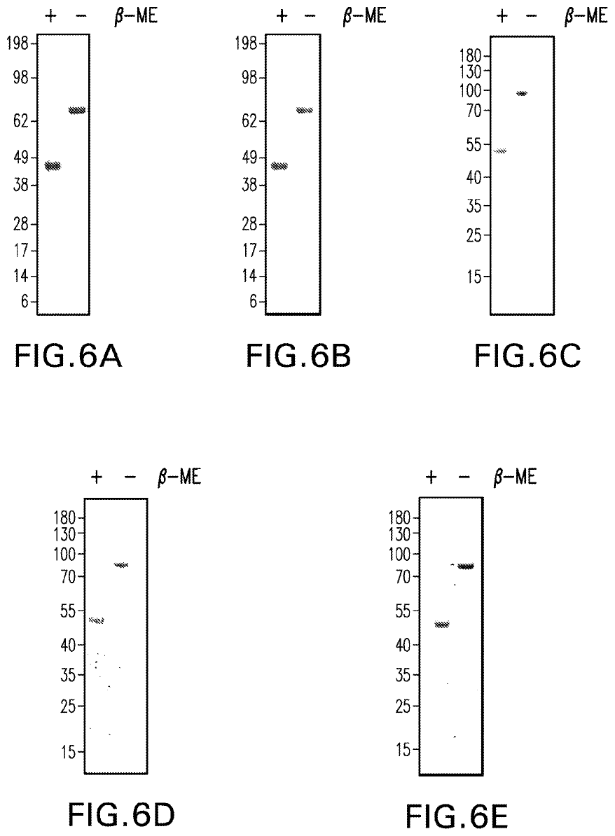

Purified VHH-Fc-AvaN constructs were concentrated down to 1 mg/ml. The ligation reaction was initiated by addition of 0.5 mM Cys-2N3 linker, 2 eq. of IntC, 100 mM of MESNa and 0.25 mM TCEP and adjusting pH to 7.5-8.0. The reaction was incubated in the dark at r.t. for 24 h and monitored by SDS-PAGE and Coomassie staining. Once the reactions were completed, they were dialyzed into PBS pH 7.4. The resulting VHH products (Isotype-VHH-Fc-2N3, PD-L1-VHH-FC-2N3) were purified by size-exclusion on an S200 column using the AKTA Purifier system. Elution from the column was monitored by UV-Vis absorbance at 280 nm. The elution volumes were in good agreement with the estimated MW. Pure fractions were pooled and concentrated down and finally analyzed by SDS-PAGE under reduced and non-reduce conditions (see FIGS. 6A-6E). The total overall yield was 40%, including thiolysis, ligation and purification.

FIGS. 6A-6E represent Coomassie stained SDS-PAGE analysis of purified Isotype-VHH-Fc-2N3 (6A), PD-L1-VHH-FC-2N3 (6B), PD-L1-VHH 2-FC-2N3 (6C), PD-L1-VHH 3-FC-2N3 (6D), and PD-1-VHH-FC-2N3 (6E) under reducing (+β-ME) and non-reducing (−β-ME) conditions.

### Example 11: LC/MS Intact Mass Analysis of Conjugates

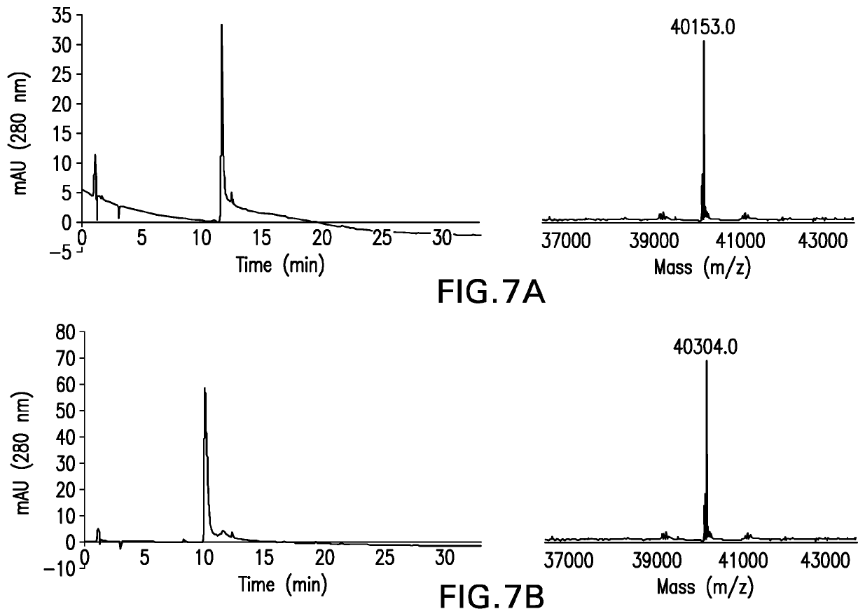

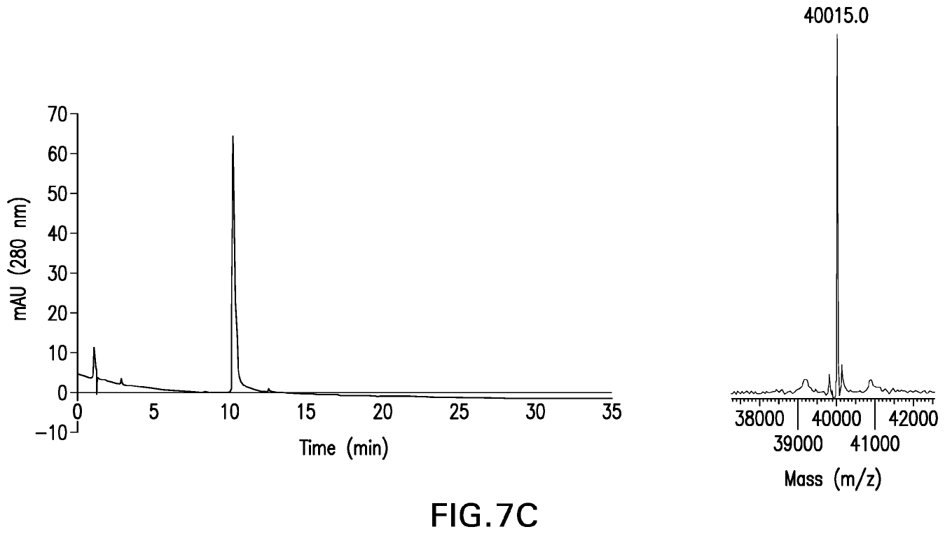

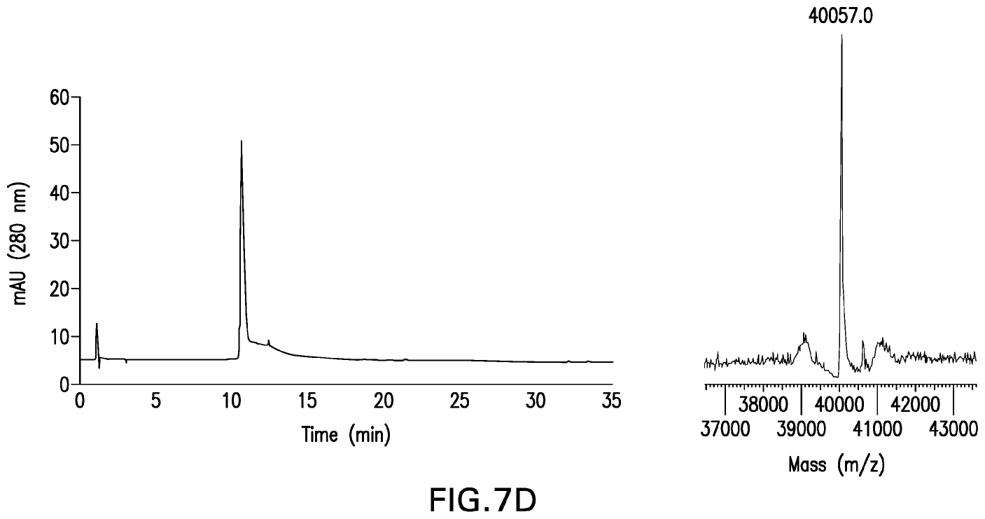

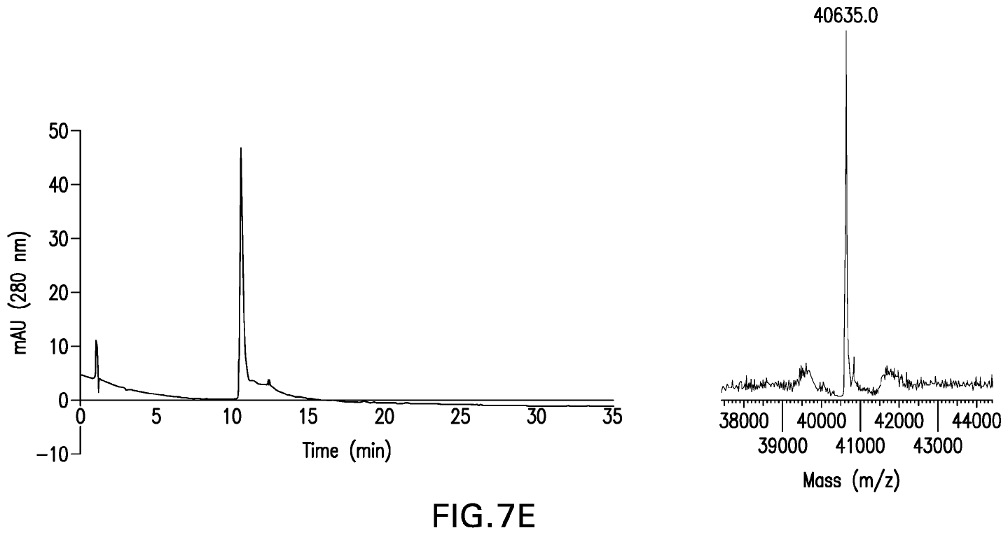

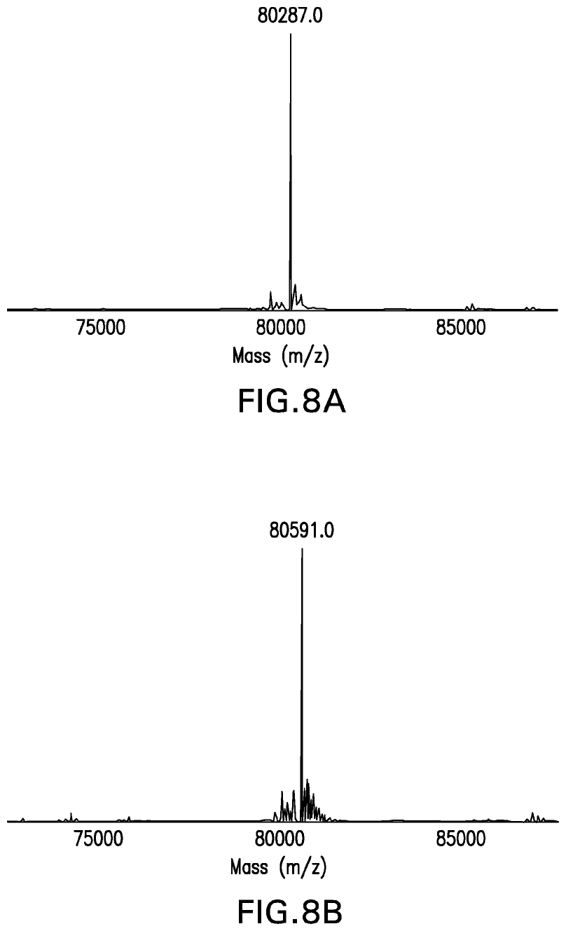

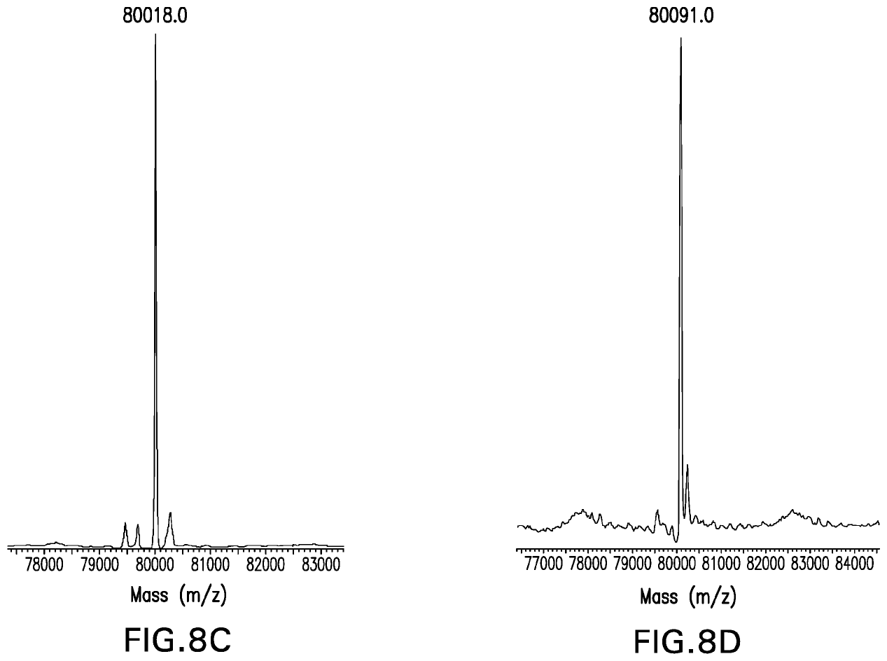

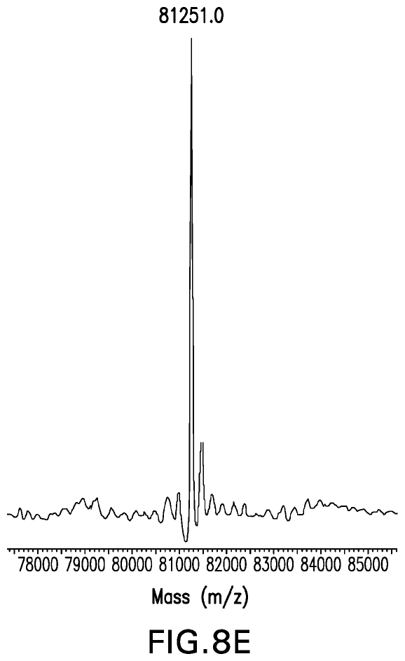

Prior to LC/MS analysis, Isotype-VHH-Fc-2N3 and PD-L1-VHH-FC-2N3 were deglycosylated with PNGase F (NEB) under non-denaturing conditions at 37° C. overnight. For analysis under reduced conditions, sample was denatured by exchanging buffer to 6 M Gn·HCl in PBS pH 7.4 and treated with 10 mM DTT at 37° C. for 1 hr. Deglycosylated and reduced samples were analyzed by RP-HPLC on a Zorbax 300SB C8 column using a 15-70% linear gradient of solvent C (0.25% Formic acid and 0.02% TFA in water) in solvent D (90% isopropanol in water with 0.25% Formic acid and 0.02 TFA) over 30 min at 1 ml/min flow rate and 70° C., preceded by a 5 minutes isocratic phase at 15% A. Peaks were collected and analyzed by electrospray ionization mass spectrometric (ESI-MS). ESI-MS analyses were performed on an LCT Premier TOF (Waters) (see Figure below). For analysis under non-reduced conditions, deglycosylated samples were analyzed directly by ESI-MS (see FIGS. 7A-7E and 8A-8E).

FIG. 7A-7E show RP-HPLC and ESI-MS analysis under reducing conditions of bis-azido conjugates. Samples were deglycosylated using PNGase F and fully reduced with DTT under denaturing conditions. RP-HPLC analysis was performed over a 15-70% B gradient on a Zorbax 300SB C8 column. FIG. 7A: RP-HPLC (left panel) and ESI-MS (right panel) for Isotype-VHH-Fc-2N3 (MW_{Obs}: 40153.0, MW_{Calc}: 40155.1), FIG. 7B: RP-HPLC (left panel) and ESI-MS (right panel) for PD-L1-VHH-Fc-2N3 (MW_{Obs}: 40304.0, MW_{Calc}: 40306.1), FIG. 7C: RP-HPLC (left panel) and ESI-MS (right panel) for PD-L1-VHH 2-Fc-2N3 (MW_{Obs}: 40015.0, MW_{Calc}: 40019.1), FIG. 7D: RP-HPLC (left panel) and ESI-MS (right panel) for PD-L1-VHH 3-Fc-2N3 (MW_{Obs}: 40057.0, MW_{Calc}: 40061.1), FIG. 7E: RP-HPLC (left panel) and ESI-MS (right panel) for PD-1-VHH-Fc-2N3 (MW_{Obs}: 40635.0, MW_{Calc}: 40637.1)

FIGS. 8A-8E show ESI-MS analysis under non-reducing conditions. Samples were deglycosylated using PNGase F and analyzed by LC-ESAI-MS. FIG. 8A: Isotype-VHH-Fc-2N3 (MW_{Obs}: 80287.0, MW_{Calc}: 80294.2), FIG. 8B: PD-L1-VHH-Fc-2N3 (MW_{Obs}: 80591.0, MW_{Calc}: 80596.2), FIG. 8C: PD-L1-VHH 2-Fc-2N3 (MW_{Obs}: 80018.0, MW_{Calc}: 80024.2), FIG. 8D: PD-L1-VHH 3-Fc-2N3 (MW_{Obs}: 80091.0, MW_{Calc}: 80098.2), FIG. 8E: PD-1-VHH-Fc-2N3 (MW_{Obs}: 81251.0, MW_{Calc}: 81253.2).

### Example 12: Preparation of α-PD-L1 mAb-N3 Conjugate (Cloning of α-PD-L1 Antibody)

PD-L1 antibody (atezolizumab, referred to as Atz) sequence was obtained from the KEGG Drug database (entry D10773, www.genome.jp/dbget-bin/www_bget?dr:D10773). The gene for the variable heavy chain (HV) and variable light chain (LV) of anti-PD-L1 antibody were purchased from Genewiz (Germany). The HV was fused to the N-terminus of the constant domain of the pFUSEN-hIgG-AvaN vector via overlap extension PCR16 to obtain the construction HC-PD-L1-AvaN. The LV was fused to the N-terminus of the kappa domain of the pFUSEN-hIgG vector via overlap extension PCR16 to obtain the construction LC-PD-L1. The resulting plasmids encoded for the following sequences:

### LC-PD-L1 Includes the Amino Acid

(SEQ ID NO: 28)
MGWSCIILFLVATATGVHSDIQMTQSPSSLSASVGDRVTITCRASQDVST
AVAWYQQKPGKAPKLLIYSASFLYSGVPSRFSGSGSGTDFTLTISSLQPE
DFATYYCQQYLYHPATFGQGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTNQGLSSPVTKSFNRGEC.

The signal peptide was represented by the following amino acid sequence: MGWSCIILFLVATATGVHS (SEQ ID NO: 24). The LV of α-PD-L1 was represented by the following amino acid sequence:

(SEQ ID NO: 29)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRT.
The Kappa domain was represented by the following
amino acid sequence:
(SEQ ID NO: 30)
VAAPSVFIFPPSDEQLKSGTASVVCLLNNFYPREAKVQWKVDNALQSGN
SQESVTEQDSKDSTYSLSSTLTLSKADYEKHKVYACEVTNQGLSSPVTK
SFNRGEC.

HC-PD-L1-AvaN represented by the following amino acid sequence:

(SEQ ID NO: 31)
MGWSCIILFLVATATGVHSEVQLVESGGGLVQPGGSLRLSCAASGFTFS
DSWIHWVRQAPGKGLEWVAWISPYGGSTYYADSVKGRFTISADTSKNTA
YLQMNSLRAEDTAVYYCARRHWPGGFDYWGQGTLVTVSSASTKGPSVFP
LAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGKASGGCLSYDTEVLTVEYGFVPIG
EIVDKGIECSVFSIDSNGIVYTQPIAQWHHRGKQEVFEYCLEDGSIIKA
TKDHKFMTQDGKMLPIDEIFEQELDLLQVKGLPEGHHHHHHG

Signal peptide is represented by the following amino acid sequence: MGWSCIILFLVATATGVHS (SEQ ID NO: 24), HV of α-PD-L1 is EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVT VSS (SEQ ID NO: 32), IgG constant domain is represented by the following amino acid sequence: ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PSVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGKASGG (SEQ ID NO: 33). AvaN intein is represented by the following amino acid sequence: CLSYDTEVLTVEYGFVPIGEIVDKGIECSVFSIDSNGIVYTQPIAQWHHRGKQEVFEYCLE DGSIIKATKDHKFMTQDGKMLPIDEIFEQELDLLQVKGLPE (SEQ ID NO: 26), and His tag is represented by the following amino acid sequence: GHHHHHHG (SEQ ID NO: 27).

### Example 13: Expression and Purification of α-PD-L1 Antibody

Expi293 cells were transiently co-transfected with LC-PD-L1 and HC-PD-L1-AvaN constructs using Expifectamine (Life Technology), according to the manufacturer's instructions. After 6 days incubation at 37° C. with 8% CO2, cell supernatants were harvested and spun down at 2000 rcf for 20 min at 4° C. After addition of complete protease inhibitors cell supernatants were dialyzed against PBS and directly purified over a Protein A column. For western blot analysis, samples were loaded onto 8% acrylamide Bis-Tris gels and run in MES-SDS running buffer. Pure fractions were pooled and stored at −80° C.

### Example 14: LC/MS Intact Mass Analysis of Conjugates

Prior to LC/MS analysis, the anti-PD-L1-2N3 antibody was deglycosylated with PNGase F (NEB) under non-denaturing conditions at 37° C. overnight. For analysis under reduced conditions, sample was denatured by exchanging buffer to 6 M Gn HCl in PBS pH 7.4 and treated with 10 mM DTT at 37° C. for 1 hr. Deglycosylated and reduced samples were analyzed by RP-HPLC on a Zorbax 300SB C8 column using a 15-70% linear gradient of solvent C (0.25% Formic acid and 0.02% TFA in water) in solvent D (90% isopropanol in water with 0.25% Formic acid and 0.02 TFA) over 30 min at 1 ml/min flow rate and 70° C., preceded by a 5 minutes isocratic phase at 15% A. Peaks were collected and analyzed by electrospray ionization mass spectrometric (ESI-MS). ESI-MS analyses were performed on an LCT Premier TOF (Waters). For analysis under non-reduced conditions, deglycosylated samples were analyzed directly by ESI-MS.

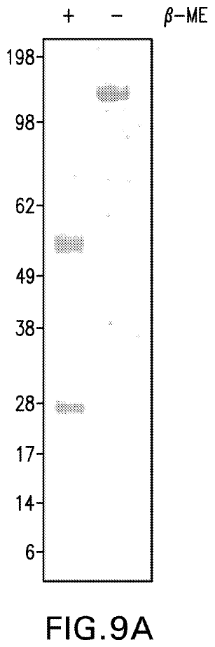

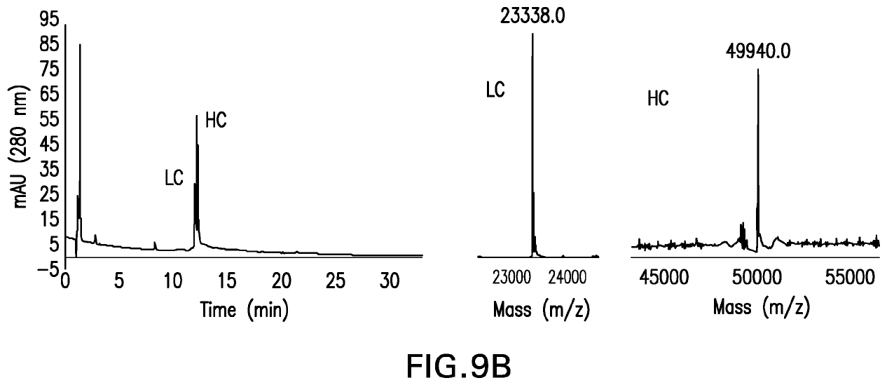

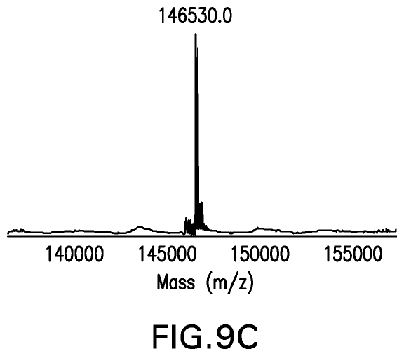

FIG. 9A depicts Coomassie stained SDSPAGE analysis of purified PDL-1-2N3 antibody (under reducing (+β-ME) and non-reducing (−β-ME) conditions. FIG. 9B depicts RP-HPLC and ESI-MS analysis under reducing conditions of bis-azido conjugates. Samples were deglycosylated using PNGase F and fully reduced with DTT under denaturing conditions. RP-HPLC analysis was performed over a 15-70% B gradient on a Zorbax 300SB C8 column. RP-HPLC (left panel) and ESI-MS (right panels) for PDL1-2N3 antibody (LC: MW_{Obs}: 23338.0, MW_{Calc}: 23341.9, HC: MW_{Obs}: 49940.0, MW_{Calc}: 49943.5). FIG. 9C depicts ESI-MS analysis under non-reducing conditions. Samples were deglycosylated using PNGase F and analyzed by LC-ESAI-MS. PDL1-2N3 antibody (MW_{Obs}: 146530.0, MW_{Calc}: 146536.2).

### Example 15: Cell Surface Binding with PD-L1 Antibody and VHH Fc-488 Fluorophore Conjugate

200 μl of α-PD-L1 mAb-N3 or VHH-Fc-N3 (1 mg/ml in PBS) was combined with 600 μM MB™ 488 DBCO (Click Chemistry Tools: 1190-5) and incubated for overnight at 4° C. The labeled VHH-Fc was then added to a Zeba Spin desalting column (Thermo Fisher Scientific: 87769, 2 ml column, 40,000 MWCO) and buffer exchanged into PBS, according to manufacturer's instructions. The final protein concentration of the VHH-Fc was measured using the BCA Protein Assay kit according to manufacturer's instructions (Thermo Fisher Scientific: 23227). 488 DBCO concentration was determined by measuring absorbance and comparing to a standard curve of free 488 DBCO of known concentrations. The micromolar concentration of 488 DBCO was divided by the micromolar concentration of VHH-Fc to determine the VHH-Fc:488 fluorophore ratio. The protein concentration was measured by the BCA assay and the photocatalyst concentration was obtained by measuring the absorption compared to free photocatalyst. Typical mAb/VHH:fluorophore ratio was 1:3.

For cell surface binding using VHH-Fc-488 conjugates, 1 million JY wt, JY PD-L1, or Raji PD-L1 cells were washed with 2×1 ml cold PBS and resuspended in PBS containing Isotype VHH-Fc-488 (Isotype), α-PD-L1 VHH-Fc-488, or α-PD-1 VHH-Fc-488 conjugate and at concentration of 5 μg/ml and incubated for 1 hr at 4° C. on a rotisserie. The cells were washed 2×s with PBS. After washing, the cell pellet was resuspended in 200 μl PBS and transferred to 5 ml FACS tubes (Fisherbrand: 14-956-3D) and analyzed on a BD FACSCelesta with BD FACSDiva software. Data was analyzed using FlowJo v10 (FlowJo, LLC).

For cell surface binding using mAb conjugate, 1 million JY wt, JY PD-L1, or Raji PD-L1 cells were washed with 2×1 ml cold PBS and combined with 2.5 μg Isotype FITC (Isotype, BD Biosciences: 555748), α-PD-L1-FITC (clone MIH1, BD Biosciences: 558065), or α-PD-L1 mAb-488 (Atz clone described above) and incubated for 1 h at 4° C. on a rotisserie. The cells were washed 2×s with PBS. After washing, the cell pellet was resuspended in 200 μl PBS and transferred to 5 ml FACS tubes (Fisherbrand: 14-956-3D) and analyzed on a BD FACSCelesta with BD FACSDiva software. Data was analyzed using FlowJo v10 (FlowJo, LLC).

### Example 16: Preparation of α-PDL1 mAb and VHH-Fc-RFT Conjugates

200 μl of α-PD-L1 mAb or VHH-Fc (1 mg/ml in PBS) was combined with 600 uM RFT DBCO and incubated for overnight at 4° C. The labeled VHH-Fc was then added to a Zeba Spin desalting column (Thermo Fisher Scientific: 87769, 2 ml column, 40,000 MWCO) and buffer exchanged into PBS, according to manufacturer's instructions. The final protein concentration of the VHH-Fc was measured using the BCA Protein Assay kit according to manufacturer's instructions (Thermo Fisher Scientific: 23227). RFT concentration was determined by measuring absorbance at 450 nm (A450) and comparing to a standard curve of free RFT of known concentrations. The micromolar concentration of RFT was divided by the micromolar concentration of VHH-Fc to determine the VHH-Fc:RFT ratio. The protein concentration was measured by the BCA assay and the photocatalyst concentration was obtained by measuring the absorption compared to free photocatalyst. Typical mAb/VHH:photocatalyst ratio was 1:3.

### Example 17: Preparation of Isotype and α-PD-L1 VHH-Fc-Peroxidase Conjugates

VHH-Fc proteins were conjugated to horse radish peroxidase (HRP) using an HRP Conjugation Kit (Abcam: ab102890) according to manufacturer's instructions. Briefly, 100 μl of VHH-Fc (1 mg/ml in PBS) was mixed with 10 μl of modifier reagent and then combined with 100 μg of HRP protein and incubated at rt overnight in the dark. 10 μl of quencher solution was added to each sample and incubated for 30 min. Peroxidase labeled VHH-Fc proteins were stored at 4° C. until used in labeling experiments described above.

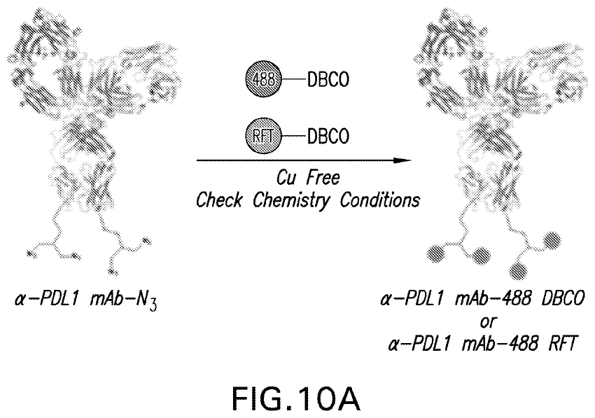

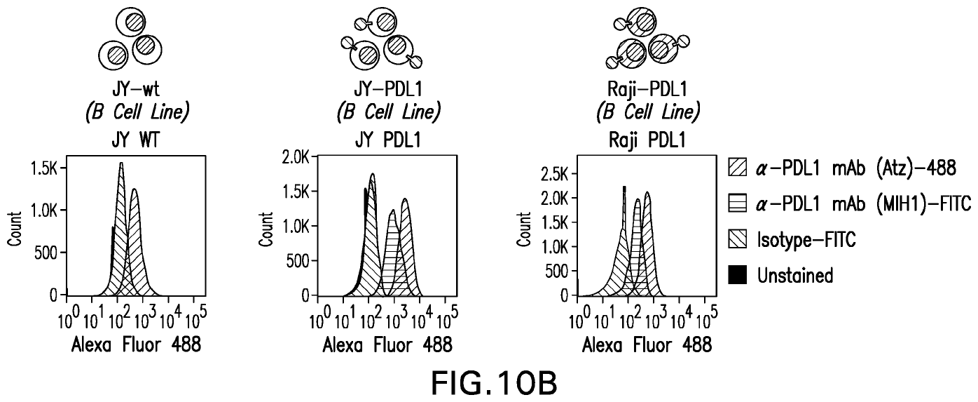

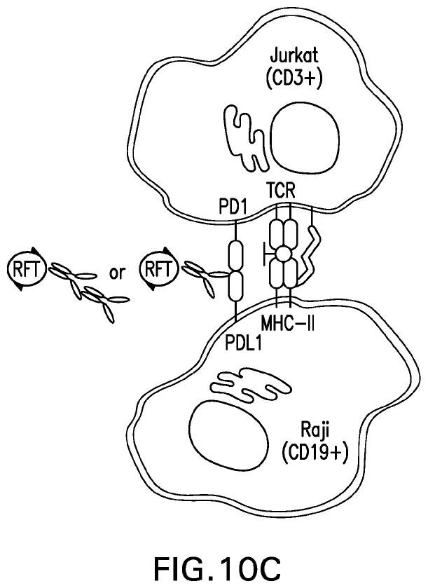

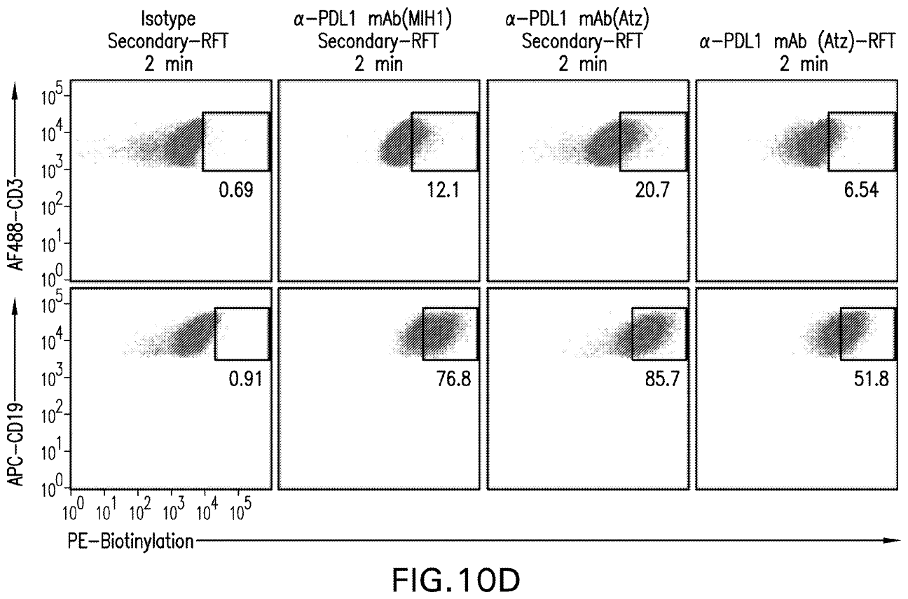

FIGS. 10A-10D depict transcellular labeling with site specific labeled α-PD-L1 mAb. FIG. 10A shows α-PD-L1 mAb (atezolizumab, Atz) is expressed as an intein fusion for C-terminal attachment of the azido linker via intein-mediated ligation to form the α-PD-L1 mAb-N3. RFT-DBCO is covalently attached to the α-PD-L1 mAb at the azido linkers to generate α-PD-L1 mAb-488 or α-PD-L1 mAb-RFT. FIG. 10B shows flow cytometry analysis shows cell-surface binding of α-PD-L1 mAb-488 or α-PD-L1 mAb-FITC (clone MIH1)) only on PD-L1 expressing cells. A non-binding control Isotype (Isotype-FITC) does not bind the cell surface.

FIG. 10C is a schematic showing targeted labeling using the PhoTag system with α-PD-L1 primary antibody and secondary antibody-RFT or α-PD-L1 mAb-RFT within a Jurkat-Raji two-cell system. FIG. 10D depicts flow cytometry analysis of the Jurkat-Raji co-culture system incubated with α-PD-L1 mAb (clone MIH1 or Atz) and secondary RFT antibody shows increased biotinylation on both Raji and Jurkat cells at 2 min light irradiation compared to isotype targeting. Targeted labeling using α-PD-L1 mAb-RFT results in lower cis and trans labeling compared to the primary/secondary antibody system. This phenomenon is likely due to the reduced number of photocatalysts present on the single antibody (˜3) compared to the secondary antibody (˜6) and that multiple secondary antibodies can likely bind to the primary antibody increasing the number of photocatalysts near PD-L1.

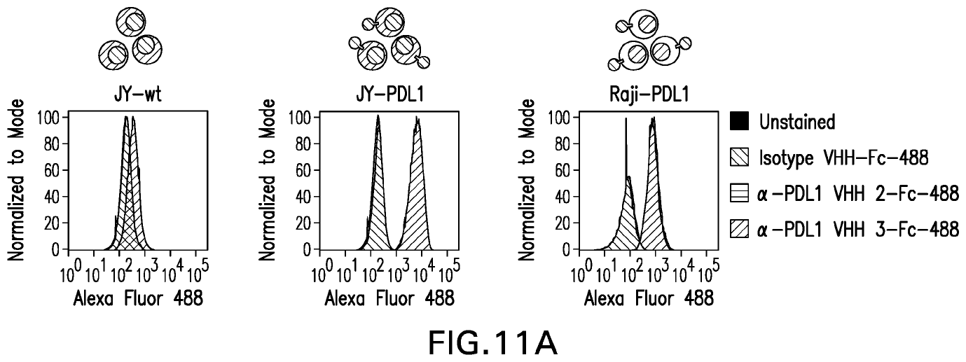

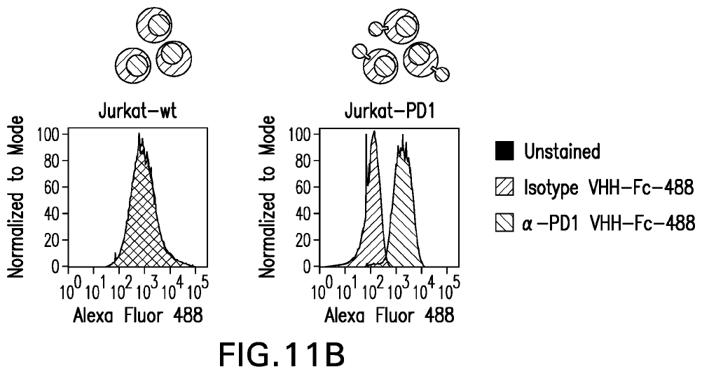

FIGS. 11A and 11B depict cell surface binding of α-PD-L1 VHH 2-Fc, α-PD-L1 VHH 3-Fc, and α-PD-1 VHH-Fc. Flow cytometry analysis shows selective cell-surface binding of VHH-Fc conjugates. FIG. 11A shows cell surface binding on JY wt, JY PD-L1, Raji PD-L1 cells with α-PD-L1 VHHs (α-PD-L1 VHH 2-Fc, α-PD-L1 VHH 3-Fc labeled with 488 dye). Compared to Unstained or Isotype VHH controls, cell surface binding for the α-PD-L1 VHHs was only detected on JY and Raji cells expressing PD-L1. FIG. 11B shows cell surface binding on Jurkat wt or Jurkat PD1 cells with α-PD-1 VHH-Fc labeled with 488 dye. As shown in the figures, compared to Unstained or Isotype VHH controls, cell surface binding was only detected on Jurkat-PD1 cells using the α-PD-1 VHH.

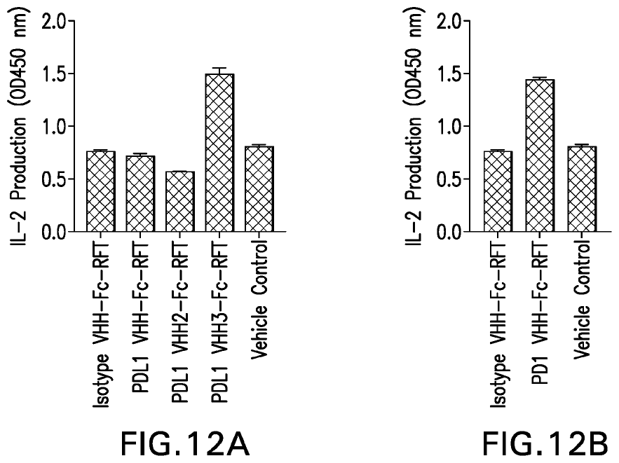

FIGS. 12A and 12B depict the effect of α-PD-L1 VHH-Fc on IL-2 Production. Raji PD-L1 cells were treated with staphylococcal enterotoxin D (SED) for 30 min and combined with Jurkat PD-1 cells pre-mixed with a) Isotype VHH-Fc-RFT, α-PD-L1 VHH-Fc-RFT, α-PD-L1 VHH 2-Fc-RFT, α-PD-L1 VHH 3-Fc-RFT, or vehicle control, or b) Isotype VHH-Fc-RFT, α-PD-1 VHH-Fc-RFT or vehicle control (10 pg/ml final concentration for VHH-Fc proteins) and incubated for 24 hours followed by analysis of IL-2 production. Bar plots of IL-2 production (as measured by an increase in OD450) show IL-2 production levels. As shown in FIG. 12A, only the blocking α-PD-L1 VHH-Fc (α-PD-L1 VHH 3-Fc-RFT) results in increased IL-2 production. In contrast, FIG. 12B shows that α-PD-1 VHH-Fc-RFT shows increased IL-2 production likely through partial blockade of the PD-1/PD-L1 interaction, which is consistent with the results as discussed with regard to FIG. 5). Error bars represent standard deviation of n=3 experiments.

### Example 18: Cell Receptor Surface Density Determination for PD-1 and PD-L1 Overexpression Cells

2 million Jurkat PD-1, Raji PD-L1, or JY PD-L1 cells were resuspended in 100 μl of PBS followed by addition of FITC α-human PD-1 (BD Biosciences: 557860) for Jurkat PD-1 cells or FITC α-human PD-L1 (BD Biosciences: 558065) for Raji PD-L1 or JY PD-L1 cells for cell surface staining. Antibody amounts were added to achieve cell surface saturation (determined empirically for each antibody). After adding antibody, the cells were incubated for 45 min at 4° C. in the dark and then washed 2×s with 500 μl of PBS. Cells were then resuspended in 250 μl Stain buffer (BD: 554656) and the geometric mean fluorescence intensity (MFI) of the cell surface antigens was measured using a BD FACSCelesta with BD FACSDiva software (v8.0.1.1). Data was analyzed using FlowJo v10 (FlowJo, LLC). Geometric MFI values were converted into antibody binding capacity values (which correlate to cell receptor surface density at antibody saturation) using the Quantum Simply Cellular u-Mouse IgG microbead kit (Bangs Laboratories, 815) according to manufacturer's instructions. Cell surface numbers were determined to be the following:
Jurkat PD-1: 323,870+/−5060 copies of PD-1/cell
Raji PD-L1: 150,440+/−2180 copies of PD-L1/cell
JY PD-L1: 650,170+/−2210 copies of PD-L1/cell

### Example 19: IL-2 Measurement in Two-Cell System to Test for PD-L1 VHH Blocking

45 μl of a 8e6 cells/ml stock of Jurkat PD-1 cells in assay media (RPMI 1640, Corning: 10-040-CV+10% dialyzed FBS, HyClone: SH30079.03) were mixed with 45 μl of vehicle control, or Isotype VHH-Fc-RFT (1:3 VHH-Fc to RFT), α-PD-L1 VHH-Fc-RFT (1:3 VHH-Fc to RFT), mouse IgG1κ isotype control antibody, clone MOPC-21 (BD Biosciences: 556648), or u-human PD-L1 antibody, clone MIH1 (Invitrogen: 14-5983-82) at a final concentration of 10 μg/ml in assay medium and incubated for 30 min at RT in a 96-well U-bottom plate. A final concentration of 120 ng/ml of Staphylococcal Enterotoxin D (SED) (Toxin Technology: DT303) diluted in assay medium was added to 8e5 cells/ml stock of Raji PD-L1 cells resuspended in assay medium was incubated for 30 min at 37° C.+5% CO₂.

After the 30 min incubation, 125 μl of SED-loaded Raji PD-L1 cells were aliquoted per well in a separate 96-well U-bottom plate and mixed with 25 μl of antibody-loaded Jurkat PD-1 cells (100,000 cells per cell line). The plate was incubated for 24 hours at 37° C.+5% CO₂. The following day, the plate was centrifuged for 1 min at 1,100 RPM and RT. 50 μl of culture supernatant per well were used to perform an ELISA using a Human IL-2 ELISA kit (Thermo Fisher: EH2IL25) per manufacturer's instructions. Absorbance measurements at 550 nm were subtracted from measurements at 450 nm and the final values were used as a readout for IL-2 production. Data was analyzed and graphed using MS Excel and GraphPad Prism software (8.1.1.330).

As shown in FIGS. 12A and 12B, the α-PD-L1 VHH-Fc does not block the PD-1/PD-L1 interaction. Raji-PD-L1 cells were treated with staphylococcal enterotoxin D (SED) for 30 min and combined with Jurkat-PD-1 cells pre-mixed with Isotype VHH-Fc-RFT, α-PD-L1 VHH-Fc-RFT, Isotype mAb, or α-PD-L1 mAb and incubated for 24 hours followed by analysis of IL-2 production. Bar plots of IL-2 production (as measured by an increase in OD450) show that only the blocking α-PD-L1 mAb (clone MIH1) results in increased IL-2 production through disruption of the PD-1/PD-L1 interaction whereas the α-PD-L1 VHH does not have blocking effects. Error bars represent standard deviation of n=3 experiments.

### Example 20: Use of α-PD-L1 VHH-Fc to Detect PD-L1 in the Immune Synapse

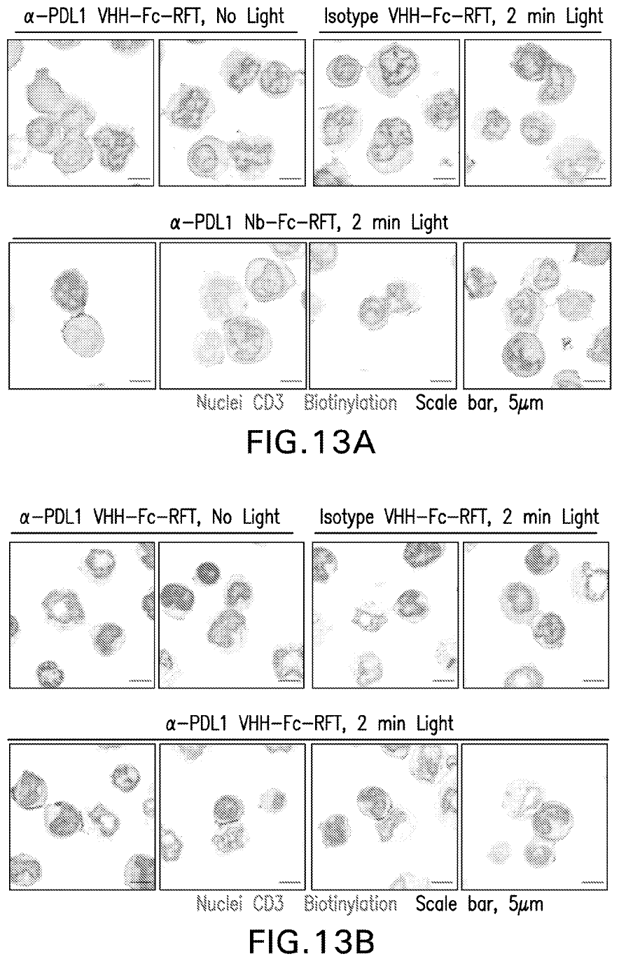

As shown in FIGS. 13A-13B, confocal microscopy imaging of Jurkat PD-1/JY PD-L1 (see FIG. 13A) or Jurkat PD-1/CHO PD-L1 (see FIG. 13B) two cell systems with α-PD-L1 VHH-Fc-RFT-targeted biotinylation on JY PD-L1 cells in the presence of 2 min visible light irradiation results in selective synaptic biotinylation whereas no biotinylation occurs in the absence of light or with isotype targeting. Cells were imaged for biotinylation (green), CD3 surface expression (magenta), and nuclei (Hoechst stain, blue). Scale bars indicate 5 μm.

1. A binding protein comprising a heavy chain antibody variable domain (VHH) that specifically binds to PD-1 or PD-L1, wherein the VHH comprises three complementarity determining regions (CDRs) selected from the group consisting of:
a) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence GSIDSRQPYDSTRRYDY (SEQ ID NO: 3);
b) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6);
c) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9);
d) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and
e) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence GSRVFDSRWYDVNEYYY (SEQ ID NO: 15).

2. The binding protein of claim 1 wherein the VHH comprises:
(a) a heavy chain variable region having at least 90% amino acid sequence identity to the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16);
(b) a heavy chain variable region having at least 90% amino acid sequence identity to the amino acid sequence: EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17);
(c) a heavy chain variable region having at least 90% amino acid sequence identity the amino acid sequence: EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or
(d) a heavy chain variable region having at least 90% amino acid sequence identity to the amino acid sequence: EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

3. The binding protein of claim 2, wherein the VHH comprises:
a) the amino acid sequence EVQLVESGGG LVQAGGSLSV SCAPSGRTFS NYAMGWFRQA PGKEREFVAR ISGGGGYTAY ADSVKGRFTI ARDNAKNTVY LQMNSLKPED TAVYYCAAGS IDSRQPYDST RRYDYWGQGT LVTVSSAAAD YKDHDGDYKD HDIDYKDDDD KGAAHHHHHH (SEQ ID NO: 16);
b) the amino acid sequence EVQLVESGGG LKQAGGSLRL SCTASARAST YIAMAWFRRT PGKAREFVAA ITWSGGHTTY ADSMKGRFTI SRDNAKNTVY LHLNALQPED AGVYYCAANQ RNTVGPSEGA YPYWGQGTLV TVSSAAADYK DHDGDYKDHD IDYKDDDDKG AAHHHHHH (SEQ ID NO: 17);
c) the amino acid sequence EVQLVESGGG LVQPGGSLRL SCAASGRTLS NHAMHWFRQA PGKEREFVSA ITWSDGETYY EDSVKGRFTI SRDNAKDTAY LEMQSLKPED TAVYYCAAKM GGPTSIPGLV EYWGQGTLVT VSSAAADYKD HDGDYKDHDI DYKDDDDKGA AHHHHHH (SEQ ID NO: 18); or
d) the amino acid sequence EVQLVESGGG LVQAGDSLRL SCVASGRTFS SYHMGWFRQA PGKEREFVAA IPRSGSNIGY SAFVKDRGTI SRDNAKNTVY LQINNLAPDD TAVYYCAAKS AAGYYSGVVF TADYDYTYWG QGTLVTVSSA AADYKDHDGD YKDHDIDYKD DDDKGAAHHH HHH (SEQ ID NO: 19).

4. The binding protein of claim 3, further comprising a signal peptide comprising the amino acid sequence: MGWSCIILFLVATATGVHS (SEQ ID NO: 24).

5. (canceled)

6. The binding protein of claim 4, fused to an Fc-domain comprising the amino acid sequence
(SEQ ID NO: 25)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGKASGG.

7. (canceled)

8. The binding protein of claim 6, further comprising an intein comprising the amino acid sequence:
(SEQ ID NO: 26)
CLSYDTEVLTVEYGFVPIGEIVDKGIECSVFSIDSNGIVYTQPIAQWHH
RGKQEVFEYCLEDGSIIKATKDHKFMTQDGKMLPIDEIFEQELDLLQVK
GLPE.

9. (canceled)

10. The binding protein of claim 8, further comprising a histidine tag comprising the amino acid sequence GHHHHHHG (SEQ ID NO: 27).

11. (canceled)

12. A VHH-Fc fusion protein comprising the amino acid sequence:
(SEQ ID NO: 20)
a) MGWSCIILFLVATATGVHSEVQLVESGGGLVQAGGSLSVSCAPSGRTFSNYAM
GWFRQAPGKEREFVARISGGGGYTAYADSVKGRFTIARDNAKNTVYLQMNSLK
PEDTAVYYCAAGSIDSRQPYDSTRRYDYWGQGTLVTVSSAAADKTHTCPPCPAP
ELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGKA
SGGCLSYDTEVLTVEYGFVPIGEIVDKGIECSVFSIDSNGIVYTQPIAQWHHRGKQ
EVFEYCLEDGSIIKATKDHKFMTQDGKMLPIDEIFEQELDLLQVKGLPEGHHHHHHG;
(SEQ ID NO: 21)
b) MGWSCIILFLVATATGVHSEVQLVESGGGLKQAGGSLRLSCTASARASTYIAMA
WFRRTPGKAREFVAAITWSGGHTTYADSMKGRFTISRDNAKNTVYLHLNALQP
EDAGVYYCAANQRNTVGPSEGAYPYWGQGTLVTVSSAAADKTHTCPPCPAPEL
LGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
ASGGCLSYDTEVLTVEYGFVPIGEIVDKGIECSVFSIDSNGIVYTQPIAQWHHRGK
QEVFEYCLEDGSIIKATKDHKFMTQDGKMLPIDEIFEQELDLLQVKGLPEGHHH
HHHG;
(SEQ ID NO: 22)
c) MGWSCIILFLVATATGVHSEVQLVESGGGLVQPGGSLRLSCAASGRTLSNHAMH
WFRQAPGKEREFVSAITWSDGETYYEDSVKGRFTISRDNAKDTAYLEMQSLKPE
DTAVYYCAAKMGGPTSIPGLVEYWGQGTLVTVSSAAADKTHTCPPCPAPELLG
GPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGKASG
GCLSYDTEVLTVEYGFVPIGEIVDKGIECSVFSIDSNGIVYTQPIAQWHHRGKQEV
FEYCLEDGSIIKATKDHKFMTQDGKMLPIDEIFEQELDLLQVKGLPEGHHHHHHG;
or
(SEQ ID NO: 23)
d) MGWSCIILFLVATATGVHSEVQLVESGGGLVQAGDSLRLSCVASGRTFSSYHMG
WFRQAPGKEREFVAAIPRSGSNIGYSAFVKDRGTISRDNAKNTVYLQINNLAPD
DTAVYYCAAKSAAGYYSGVVFTADYDYTYWGQGTLVTVSSAAADKTHTCPPC
PAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLS
PGKASGGCLSYDTEVLTVEYGFVPIGEIVDKGIECSVFSIDSNGIVYTQPIAQWHH
RGKQEVFEYCLEDGSIIKATKDHKFMTQDGKMLPIDEIFEQELDLLQVKGLPEGH
HHHHHG.

13. A nucleic acid molecule comprising a sequence of nucleotides that encodes the binding protein of claim 6.

14. A polypeptide comprising the V_{H} domains of any of the binding proteins of claim 3.

15. An isolated nucleic acid encoding the V_{H} domains of any of the binding proteins of claim 3.

16. An expression vector comprising the isolated nucleic acid of claim 15.

17. A host cell comprising the expression vector of claim 16.

18. A method of producing a binding protein, comprising:
a) culturing a host cell comprising an expression vector that comprises the nucleic acid molecule that encodes the binding protein of claim 6 in culture medium under conditions wherein the nucleic acid sequence is expressed, thereby producing a polypeptide comprising the heavy chain variable regions and
b) recovering the polypeptides from the host cell or culture medium.

19. A method of preparing a PD-1 or PD-L1 conjugate comprising the steps of:
a) providing a PD-1 or PD-L1 binding protein according to claim 3;
b) optionally, providing a fusion protein comprising an Fc-domain comprising an amino acid sequence selected from SEQ ID NOs: 20, 21, 22, or 23;
c) providing an intein; and
d) fusing the intein to the C-terminus of the PD-1 or PD-L1 binding protein to form a PD-1 or PD-L1-intein conjugate or, optionally, fusing the intein to the C-terminus of the fusion protein to form a PD-1 or PD-L1-fusion protein-intein conjugate.

20. A method for detecting cell-cell interactions, the method comprising:
a) contacting a first unlabeled cell with a VHH-photocatalyst conjugate to form a first labeled cell, wherein the VHH-photocatalyst conjugate comprises a VHH having three complementarity determining regions (CDRs) selected from the group consisting of:
i) a CDR1 comprising the amino acid sequence GRTFSNYAMG (SEQ ID NO: 1), a CDR 2 comprising the amino acid sequence RISGGGGYTAYADSVKG (SEQ ID NO: 2), and a CDR 3 comprising the amino acid sequence
(SEQ ID NO: 3)
GSIDSRQPYDSTRRYDY;
ii) a CDR1 comprising the amino acid sequence RASTYIAMA (SEQ ID NO: 4), a CDR 2 comprising the amino acid sequence AITWSGGHTTYADSMKG (SEQ ID NO: 5), and a CDR 3 comprising the amino acid sequence NQRNTVGPSEGAYPY (SEQ ID NO: 6);
iii) a CDR1 comprising the amino acid sequence GRTLSNHAMH (SEQ ID NO: 7), a CDR 2 comprising the amino acid sequence AITWSDGETYYEDSVKG (SEQ ID NO: 8), and a CDR 3 comprising the amino acid sequence KMGGPTSIPGLVEY (SEQ ID NO: 9);
iv) a CDR1 comprising the amino acid sequence GRTLSNHAMH (SEQ ID NO: 10), a CDR 2 comprising the amino acid sequence AIPRSGSNIGYSAFVKD (SEQ ID NO: 11), and a CDR 3 comprising the amino acid sequence and KSAAGYYSGVVFTADYDYTY (SEQ ID NO: 12); and
v) a CDR1 comprising the amino acid sequence or GLTFSVYRMG (SEQ ID NO: 13), a CDR 2 comprising the amino acid sequence AISRIADSTYYADSVKG (SEQ ID NO: 14), and a CDR 3 comprising the amino acid sequence
(SEQ ID NO: 15)
GSRVFDSRWYDVNEYYY;
b) providing a second unlabeled cell;
c) combining the first labeled cell and the second unlabeled cell to form a cell-cell conjugate system;
d) applying visible light in the range of approximately about 400-800 nm to the cell-cell conjugate system; and
e) administering a labeling probe to the cell-cell conjugate system;
wherein the labeling probe covalently attaches to the first labeled cell and the second unlabeled cell in the cell-cell conjugate system; and
f) detecting the labeling probe in the cell-cell conjugate system, wherein detection of the labeling probe indicates a cell-cell interaction between the first labeled cell and the second unlabeled cell.

21. The method of claim 20, wherein applying visible light activates the VHH-photocatalyst.

22. (canceled)

23. The method of claim 20, wherein the VHH-photocatalyst activates the labeling probe.

24. (canceled)

25. The method of claim 20, wherein the first unlabeled cell comprises PD-L1 or PL-1.

26. (canceled)

27. The method of claim 20, wherein the second unlabeled cell comprises PD-L1 or PD-1.

28. (canceled)

29. The method of claim 20, wherein the VHH-photocatalyst conjugate does not block PD-1/PD-L1 interaction of the cell.

30. The method of claim 20, wherein the VHH-photocatalyst conjugate binds to PD-L1 at the cell surface, but does not disrupt PD-1/PD-L1-mediated inhibition of IL-2 production.

31. (canceled)
